SEQUENCE LISTING (1 sequence)

SEQ ID NO: 1 - protein, 191 aa, homo sapiens
FPTIPLSRLFDDAMLRAHRLHQLAFDTYQEFEEAYIPKEQKYSFLQDPQTSLCFSESIPTPSDREETQQKSDLELLRISLLLIQSWLEPVQSLRSVFADSLVYGASDSDVYDLLKDLEEGIQTLMGRLEDGSPRTGQIFKQTYSKFDTDSHDDDALLKDYGLLYCFRKDMDKVETFLRIVQCRSVEGSCGF

(19) United States
(12) Patent Application Publication
(10) Pub. No.: US 2004/0038892 A1
(43) Pub. Date: Feb. 26, 2004

(54) Chemically-modified human growth hormone conjugates

(76) Inventors: Rory Finn, Manchester, MO (US); Wei Liao, Chesterfield, MO (US); Ned Siegel, Belleville, IL (US)

Correspondence Address: Pharmacia Corporation, Global Patent Department, P. O. Box 1027, Mail Zone MC5, St. Louis, MO, 63141, US

(21) Appl. No.: 10/441,985

(22) Filed: May 20, 2003

Related U.S. Application Data
(63) Continuation-in-part of application No. 10/300,822, filed on Nov. 20, 2002.
(60) Provisional application No. 60/331,907, filed on Nov. 20, 2001.

(51) Int. Cl. A61K 38/27; C07K 14/61

(52) U.S. Cl. 514/12; 530/399

(57) ABSTRACT

The present invention provides a chemically modified human Growth Hormone (hGH) prepared by binding a water soluble polymer to the protein. The chemically-modified protein according to the present invention may have a much longer lasting hGH activity than that of the un-modified hGH, enabling reduced dose and scheduling opportunities.

The present application is a continuation in part of U.S. application Ser. No. 10/300,822, filed Nov. 20, 2002, which claimed priority under Title 35, United States Code, §119 to U.S. Provisional application Serial No. 60/331,907, filed Nov. 20, 2001, which are incorporated by reference in their entirety as if written herein.

### FIELD OF THE INVENTION

The present invention relates to a chemical modification of human Growth Hormone (hGH) and agonist variants thereof by which the chemical and/or physiological properties of hGH can be changed. The PEGylated hGH may have an increased plasma residency duration, decreased clearance rate, improved stability, decreased antigenicity, or a combination thereof. The present invention also relates to processes for the modification of hGH. In addition, the present invention relates to pharmaceutical compositions comprising the modified hGH. A further embodiment is the use of the modified hGH for the treatment of growth and development disorders.

### BACKGROUND OF THE INVENTION

Human growth hormone (hGH) is a protein comprising a single chain of 191 amino acids cross-linked by two disulphide bridges and the monomeric form has a molecular weight of 22 kDa. Human GH is secreted by the pituitary gland and which also can be produced by recombinant genetic engineering. hGH will cause growth in all bodily tissues that are capable of growth. Recombinant hGH has been commercially available for several years. Two types of therapeutically useful recombinant hGH preparations are present on the market: the authentic one, e.g. Genotropin™, or Nutropin™ and an analogue with an additional methionine residue at the N-terminal end, e.g. Somatonorm™. hGH is used to stimulate linear growth in patients with hypo pituitary dwarfism also referred to as Growth Hormone Deficiency (GHD) or Turner's syndrome but other indications have also been suggested including long-term treatment of growth failure in children who were born short for gestational age (SGA), for treatment of patients with Prader-Willi syndrome (PWS), chronic renal insufficiency (CRI), Aids wasting, and Aging.

A major biological effect of growth hormone (GH) is to promote growth in young mammals and maintenance of tissues in older mammals. The organ systems affected include the skeleton, connective tissue, muscles, and viscera such as liver, intestine, and kidneys. Growth hormones exert their effect through interaction with specific receptors on the target cell's membrane. hGH is a member of a family of homologous hormones that include placental lactogens, prolactins, and other genetic and species variants or growth hormone (Nicoll, C. S., et al. (1986) Endocrine Reviews 7: 169). hGH is unusual among these in that it exhibits broad species specificity and binds to either the cloned somatogenic (Leung, D. W., et al. [1987] Nature 330; 537) or prolactin receptor (Boutin, J. M., et al. [1988] Cell; 53: 69). The cloned gene for hGH has been expressed in a secreted form in Escherichia coli (Chang, C. N., et al. [1987] Gene 55:189), and its DNA and amino acid sequence has been reported (Goeddel, et al. [1979) Nature 281: 544; Gray, et al. [1985] Gene 39:247).

Human growth hormone (hGH) participates in much of the regulation of normal human growth and development. This pituitary hormone exhibits a multitude of biological effects including linear growth (somatogenesis), lactation, activation of macrophages, insulin-like and diabetogenic effects among others (Chawla, R, K. (1983) Ann. Rev. Med. 34, 519; Edwards, C. K. et al. (1988) Science 239, 769; Thomer, M. O., et al. (1988) J. Clin. Invest. 81:745). Growth hormone deficiency in children leads to dwarfism, which has been successfully treated for more than a decade by exogenous administration of hGH.

Human growth hormone (hGH) is a single-chain polypeptide consisting of 191 amino acids (molecular weight 21,500). Disulfide bonds link positions 53 and 165 and positions 182 and 189. Niall, Nature, New Biology, 230:90 (1971). hGH is a potent anabolic agent, especially due to retention of nitrogen, phosphorus, potassium, and calcium. Treatment of hypophysectomized rats with GH can restore at least a portion of the growth rate of the rats. Moore et al., Endocrinology 122:2920-2926 (1988). Among its most striking effects in hypo pituitary (GH-deficient) subjects is accelerated linear growth of bone-growth-plate-cartilage resulting in increased stature. Kaplan, Growth Disorders in Children and Adolescents (Springfield, Ill.: Charles C. Thomas, 1964.

hGH causes a variety of physiological and metabolic effects in various animal models including linear bone growth, lactation, activation of macrophages, insulin-like and diabetogenic effects, and others (R. K. Chawla et al., Annu. Rev. Med. 34:519 (1983); O. G. P. Isaksson et al., Annu. Rev. Physiol. 47, 483 (1985); C. K. Edwards et al., Science 239, 769 (1988); M. O. Thomer and M. L. Vance, J. Clin. Invest. 82:745 (1988); J. P. Hughes and H. G. Friesen, Ann. Rev. Physiol. 47:469 (1985)). It has been reported that, especially in women after menopause, GH secretion declines with age. Millard et al., Neurobiol. Aging, 11:229-235 (1990); Takahashi et al., Neuroendocrinology M, L6-137-142 (1987). See also Rudman et al., J. Clin. Invest., 67:1361-1369 (1981) and Blackman, Endocrinology and Aging, 16:981 (1987). Moreover, a report exists that some of the manifestations of aging, including decreased lean body mass, expansion of adipose-tissue mass, and the thinning of the skin, can be reduced by GH treatment three times a week. See, e.g., Rudman et al., N. Eng. J. Med., 323:1-6 (1990) and the accompanying article in the same journal issue by Dr. Vance (pp. 52-54). These biological effects derive from the interaction between hGH and specific cellular receptors. Two different human receptors have been cloned, the hGH liver receptor (D. W. Leung et al., Nature 330:537(1987)) and the human prolactin receptor (J. M. Boutin et al., Mol. Endocrinology. 3:1455 (1989)). However, there are likely to be others including the human placental lactogen receptor (M. Freemark, M. Comer, G. Komer, and S. Handwerger, Endocrinol. 120:1865 (1987)). These homologous receptors contain a glycosylated extracellular hormone binding domain, a single transmembrane domain, and a cytoplasmic domain, which differs considerably in sequence and size. One or more receptors are assumed to play a determining role in the physiological response to hGH.

It is generally observed that physiologically active proteins administered into a body can show their pharmacological activity only for a short period of time due to their high clearance rate in the body. Furthermore, the relative hydrophobicity of these proteins may limit their stability and/or solubility.

For the purpose of decreasing the clearance rate, improving stability or abolishing antigenicity of therapeutic proteins, some methods have been proposed wherein the proteins are chemically modified with water-soluble polymers. Chemical modification of this type may block effectively a proteolytic enzyme from physical contact with the protein backbone itself, thus preventing degradation. Chemical attachment of certain water-soluble polymers may effectively reduce renal clearance due to increased hydrodynamic volume of the molecule. Additional advantages include, under certain circumstances, increasing the stability and circulation time of the therapeutic protein, increasing solubility, and decreasing immunogenicity. Poly(alkylene oxide), notably poly(ethylene glycol) (PEG), is one such chemical moiety that has been used in the preparation of therapeutic protein products (the verb “pegylate” meaning to attach at least one PEG molecule). The attachment of poly(ethylene glycol) has been shown to protect against proteolysis, Sada, et al., J. Fermentation Bioengineering 71: 137-139 (1991), and methods for attachment of certain poly(ethylene glycol) moieties are available. See U.S. Pat. No. 4,179,337, Davis et al., “Non-Immunogenic Polypeptides,” issued Dec. 18, 1979; and U.S. Pat. No. 4,002,531, Royer, “Modifying enzymes with Polyethylene Glycol and Product Produced Thereby,” issued Jan. 11, 1977. For a review, see Abuchowski et al., in Enzymes as Drugs. (J. S. Holcerberg and J. Roberts, eds. pp. 367-383 (1981)).

Other water-soluble polymers have been used, such as copolymers of ethylene glycol/propylene glycol, poly(oxyethylated polyol), poly(olefenic alcohol), poly(acryloyl morpholine), poly(oxazoline), poly-(hydroxyethyl methacrylate), carboxymethylcellulose, dextran, poly(vinyl alcohol), poly(vinyl pyrrolidone), poly(-1,3-dioxolane), poly(-1,3,6-trioxane), ethylene/maleic anhydride copolymer, polyamino acids (either homopolymers or random copolymers).

A number of examples of pegylated therapeutic proteins have been described. ADAGEN®, a pegylated formulation of adenosine deaminase, is approved for treating severe combined immunodeficiency disease. ONCASPAR®, a pegylated L-asparaginase has been approved for treating hypersensitive ALL patients. Pegylated superoxide dismutase has been in clinical trials for treating head injury. Pegylated α-interferon (U.S. Pat. Nos. 5,738,846, 5,382,657) has been approved for treating hepatitis; pegylated glucocerebrosidase and pegylated hemoglobin are reported to have been in preclinical testing. Another example is pegylated IL-6, EF 0 442 724, entitled, “Modified hIL-6,” which discloses poly(ethylene glycol) molecules added to IL-6.

Another specific therapeutic protein, which has been chemically modified, is granulocyte colony stimulating factor, (G-CSF). G-CSF induces the rapid proliferation and release of neutrophilic granulocytes to the blood stream, and thereby provides therapeutic effect in fighting infection. European patent publication EP 0 401 384, published Dec. 12, 1990, entitled, “Chemically Modified Granulocyte Colony Stimulating Factor,” describes materials and methods for preparing G-CSF to which poly(ethylene glycol) molecules are attached. Modified G-CSF and analogs thereof are also reported in EP 0 473 268, published Mar. 4, 1992, entitled “Continuous Release Pharmaceutical Compositions Comprising a Polypeptide Covalently Conjugated To A Water Soluble Polymer,” stating the use of various G-CSF and derivatives covalently conjugated to a water soluble particle polymer, such as poly(ethylene glycol). A modified polypeptide having human granulocyte colony stimulating factor activity is reported in EP 0 335 423 published Oct. 4, 1989. Provided in U.S. Pat. No. 5,824,784 are methods for N-terminally modifying proteins or analogs thereof, and resultant compositions, including novel N-terminally chemically modified G-CSF compositions. U.S. Pat. No. 5,824,778 discloses chemically modified G-CSF.

For poly(ethylene glycol), a variety of means have been used to attach the poly(ethylene glycol) molecules to the protein. Generally, poly(ethylene glycol) molecules are connected to the protein via a reactive group found on the protein.

Amino groups, such as those on lysine residues or at the N-terminus, are convenient for such attachment. For example, Royer (U.S. Pat. No. 4,002,531, above) states that reductive alkylation was used for attachment of poly(ethylene glycol) molecules to an enzyme. EP 0 539 167, published Apr. 28, 1993, Wright, “Peg Imidates and Protein Derivatives Thereof” states that peptides and organic compounds with free amino group(s) are modified with an imidate derivative of PEG or related water-soluble organic polymers. U.S. Pat. No. 5,298,643 and U.S. Pat. No. 5,637,749 disclose PEG aryl imidates

Chamow et al., Bioconjugate Chem. 5: 133-140 (1994) report the modification of CD4 immunoadhesin with monomethoxypoly(ethylene glycol) aldehyde via reductive alkylation. The authors report that 50% of the CD4-Ig was MePEG-modified under conditions allowing control over the extent of pegylation. Id. at page 137. The authors also report that the in vitro binding capability of the modified CD4-Ig (to the protein gp 120) decreased at a rate correlated to the extent of MePEGylation Ibid. U.S. Pat. No. 4,904,584, Shaw, issued Feb. 27, 1990, relates to the modification of the number of lysine residues in proteins for the attachment of poly(ethylene glycol) molecules via reactive amine groups.

Many methods of attaching a polymer to a protein involve using a moiety to act as a linking group. Such moieties may, however, be antigenic. A tresyl chloride method involving no linking group is available, but this method may be difficult to use to produce therapeutic products as the use of tresyl chloride may produce toxic by-products. See Francis et al., In: Stability of protein pharmaceuticals: in vivo pathways of degradation and strategies for protein stabilization (Eds. Ahern, T. and Manning, M. C.) Plenum, N.Y., 1991) Also, Delgado et al., “Coupling of PEG to Protein By Activation With Tresyl Chloride, Applications In Immunoaffinity Cell Preparation”, in Separations Using Aqueous Phase Systems, Applications In Cell Biology and Biotechnology, Fisher et al., eds. Plenum Press, New York, N.Y., 1989 pp. 211-213.

See also, Rose et al., Bioconjugate Chemistry 2: 154-159 (1991) which reports the selective attachment of the linker group carbohydrazide to the C-terminal carboxyl group of a protein substrate (insulin).

WO 93/00109 relates to a method for stimulating a mammal's or avian's GH responsive tissues comprising maintaining a continuous, effective plasma GH concentration for a period of 3 or more days. One way of achieving such plasma concentration is stated to be by use of GH coupled to a macromolecular substance such as PEG (polyethylene glycol). The coupling to a macromolecular substance is stated to result in improved half-life. PEGylated human growth hormone has been reported in WO 93/00109 using mPEG aldehyde-5000 and mPEG N-hydroxysuccinmidyl ester(mPEG-NHS-5000). The use of mPEG-NHS resulted in heterogeneous mixtures of multiply PEGylated forms of hGH. WO 93/00109 also discloses the use of mPEG-maleimide to PEGylate cysteine hGH variants.

WO 99/03887 discloses a cysteine variant growth hormone that is PEGylated. Designated as BT-005, this conjugate is purported to be more effective at stimulating weight gain in growth hormone deficient rats and to have a longer half-life than hGH.

PEGylated human growth hormone has also been reported in Clark et al. using succinimidyl ester of carboxymethylated PEG (Journal of Biological Chemistry 271:21969-21977, 1996). Clark et al. describes derivates of hGH of increasing size using mPEG-NHS-5000, which selectively conjugates to primary amines. Increasing levels of PEG modification reduced the affinity for its receptor and increased the EC₅₀ in a cell-based assay up to 1500 fold. Olson et al., Polymer Preprints 38:568-569, 1997 discloses the use of N-hydroxysuccinimide (NHS) PEG and succinimidyl propionate (SPA) PEG to achieve multiply PEGylated hGH species.

WO 94/20069 prophetically discloses PEGylated hGH as part of a formulation for pulmonary delivery.

U.S. Pat. No. 4,179,337 discloses methods of PEGylating enzymes and hormones to obtain physiologically active non-immunogenic, water-soluble polypeptide conjugates. GH is mentioned as one example of a hormone to be PEGylated.

EP 458064 A2 discloses PEGylation of introduced or naturally present cysteine residues in somatotropin. EP 458064 A2 further mentions the incorporation of two cysteine residues in a loop termed the omega loop stated to be located at residues 102-112 in wild type bovine somatotropin, more specifically EP 458064 A2 discloses the substitution of residues numbered 102 and 112 of bovine somatotropin from Ser to Cys and Tyr to Cys, respectively.

WO 95/11987 suggests attachment of PEG to the thiol group of a cysteine residue being either present in the parent molecule or introduced by site directed mutagenesis. WO 95/11987 relates to PEGylation of protease nexin-1, however PEGylation in general of hGH and other proteins is suggested as well.

WO 99/03887 discloses, e.g., growth hormone modified by insertion of additional cysteine for serine residues_and attachment of PEG to the introduced cysteine residues.

WO 00/42175 relates to a method for making proteins containing free cysteine residues for attachment of PEG. WO 00/42175 discloses the following muteins of hGH: T3C, S144C and T148C and the cysteine PEGylation thereof.

WO 9711178 (as well as U.S. Pat. No. 5,849,535, U.S. Pat. No. 6,004,931, and U.S. Pat. No. 6,022,711) relates to the use of GH variants as agonists or antagonists of hGH. WO 9711178 also discloses PEGylation of hGH, including lysine PEGylation and the introduction or replacement of lysine (e.g. K168A and K172R). WO 9711178 also discloses the substitution G120K.

The previous reports of PEGylated hGH require the attachment of multiple PEGs, which results in undesirable product heterogeneity, to achieve a hydrodynamic volume greater than the 70K molecular weight cut-off of the kidney filtration as described (Knauf, M. J. et al, J. Biol. Chem. 263:15064-15070,1988).

A GH molecule with a longer circulation half-life would decrease the number of necessary administrations and potentially provide more optimal therapeutic hGH levels with concomitant enhanced therapeutic effect.

The present invention provides chemically modified hGH conjugates having decreased heterogeneity, decreased clearance rate, increased plasma residency duration, improved solubility, increased stability, decreased antigenicity, or combinations thereof.

### SUMMARY OF THE INVENTION

The present invention relates to chemically modified hGH and agonist variants thereof, which have at least one improved chemical or physiological property selected from but not limited to decreased clearance rate, increased plasma residency duration, increased stability, improved solubility, and decreased antigenicity. Thus, as described below in more detail, the present invention has a number of aspects relating to chemically modifying hGH and agonist variants thereof as well as specific modifications using a variety of poly(ethylene glycol) moieties.

The present invention also relates to methods of producing the chemically modified hGH and agonist variants thereof.

The present invention also relates to compositions comprising the chemically modified hGH and agonist variants thereof.

The modified hGH and agonist variants thereof of the present invention may be useful in the treatment of, but not limited to, dwarfism (GHD), Adult GHD, Turner's syndrome, long-term treatment of growth failure in children who were born short for gestational age (SGA), for treatment of patients with Prader-Willi syndrome (PWS), chronic renal insufficiency (CRI), Aids wasting, Aging, End-stage Renal Failure, and Cystic Fibrosis.

### BRIEF DESCRIPTION OF THE DRAWINGS

FIG. 1 is a reproduction of a reducing and non-reducing SDS-PAGE analysis of the products of the reaction of hGH and 20K PEG-ALD and the anion exchange purified 20K PEG-ALD hGH. Lane 1. MW Protein standards; Lane 2. reduced hGH-10 ug; Lane 3. reduced 20 K linear PEG-ALD hGH reaction mix-10 ug; Lane 4. reduced anion exchange purified 20 K linear PEG-ALD hGH-10 ug Lane 5. Blank; Lane 6. non-reduced hGH-10 ug; Lane 7. non-reduced 20 K linear PEG-ALD hGH reaction mix-10 ug; Lane 8. non-reduced anion exchange purified 20 K linear PEG-ALD hGH-10 ug; Lane 9. Blank; Lane 10. MW Protein standards.

FIG. 2 is a reproduction of a non-reducing SDS-PAGE analysis of various anion exchange purified pegylated hGH molecules. Lane 1. MW Protein standards; Lane 2. hGH-10 ug; Lane 2. 4-6×5K PEG-SPA hGH-10 ug; Lane 3. 20 K linear PEG-ALD hGH-10 ug; Lane 4. 20 K branched PEG-ALD hGH-10 ug Lane 5. 40 K branched PEG hGH-10 ug.

FIG. 3 shows reproductions of RP-HPLC elution profiles for trypsin digests of hGH, 40K Br PEG-ALD hGH and 40K Br PEG-NHS hGH. PEG coupled primarily to the N-terminus of hGH (as shown in the 40K Br ALD hGH) results in a reduction in the N-terminal (T1) fragment peak with generation of a new PEGylated T1 peak.

FIG. 4 compares the in vivo bioactivity of unPEGylated hGH dosed daily (0.3 mg/Kg/day) to mono-PEGylated hGH dosed subcutaneously(SC) once every six days(1.8 mg/Kg) by illustrating the weight gain in hypophysectomized rats during a period of 11 days.

FIG. 5 compares the in vivo bioactivity of unPEGylated hGH dosed SC daily (0.3 mg/Kg/day) to 4-6×5K PEG-SPA-hGH, mono-PEGylated 20K branched PEG-ALD hGH, and mono-PEGylated 40K branched PEG-ALD hGH each dosed SC once every six days (1.8 mg/Kg) by illustrating the weight gain in hypophysectomized rats during a period of 11 days.

FIG. 6 compares the in vivo bioactivity of unPEGylated hGH dosed SC daily (0.3 mg/Kg/day) to 4-6×5K PEG-CMHBA-hGH, mono-PEGylated 20K linear ALD, mono-PEGylated 30K linear ALD, mono-PEGylated 20K branched PEG-ALD hGH, and mono-PEGylated 40K branched PEG-ALD hGH each dosed SC once every six days (1.8 mg/Kg) by illustrating the increase in tibial bone growth in hypophysectomized rats during a period of 11 days.

FIG. 7 compares the in vivo bioactivity of a single 1.8 mg/Kg SC dose of unPEGylated hGH, mono-PEGylated 5K linear PEG-ALD hGH, mono-PEGylated 20K linear PEG-ALD hGH, mono-PEGylated 20K branched PEG-ALD hGH, mono-PEGylated 20K linear PEG-Hydrazide hGH, mono-PEGylated 30K linear PEG-ALD hGH, mono-PEGylated 40K branched PEG-ALD hGH, 4-6×5K PEG SPA hGH, 4-6×5K PEG-CMHBA hGH by illustrating the increase in plasma IGF-1 levels in hypophysectomized rats during a period of 9 days.

### DETAILED DESCRIPTION

hGH and agonist variants thereof are members of a family of recombinant proteins, described in U.S. Pat. No. 4,658,021 and U.S. Pat. No. 5,633,352. Their recombinant production and methods of use are detailed in U.S. Pat. Nos. 4,342,832, 4,601,980; U.S. Pat. No. 4,898,830; U.S. Pat. No. 5,424,199; and U.S. Pat. No. 5,795,745.

Any purified and isolated hGH or agonist variant thereof, which is produced by host cells such as E. coli and animal cells transformed or transfected by using recombinant genetic techniques, may be used in the present invention. Additional hGH variants are described in U.S. Ser. No. 07/715,300 filed Jun.14, 1991 and Ser. No. 07/743,614 filed Aug. 9, 1991, and WO 92/09690 published Jun. 11, 1992. Among them, hGH or agonist variant thereof, which is produced by the transformed E. coli, is particularly preferable. Such hGH or agonist variant thereof may be obtained in large quantities with high purity and homogeneity. For example, the above hGH or agonist variant thereof may be prepared according to a method disclosed in U.S. Pat. Nos. 4,342,832, 4,601,980; U.S. Pat. No. 4,898,830; U.S. Pat. No. 5,424,199; and U.S. Pat. No. 5,795,745. The term “substantially has the following amino acid sequence” means that the above amino acid sequence may include one or more amino-acid changes (deletion, addition, insertion or replacement) as long as such changes will not cause any disadvantageous non-similarity in function to hGH or agonist variant thereof. It is more preferable to use the hGH or agonist variant thereof substantially having an amino acid sequence, in which at least one lysine, aspartic acid, glutamic acid, unpaired cysteine residue, a free N-terminal α-amino group or a free C-terminal carboxyl group, is included.

According to the present invention, poly(ethylene glycol) is covalently bound through amino acid residues of hGH or agonist variant thereof. A variety of activated poly(ethylene glycol)s having a number of different functional groups, linkers, configurations, and molecular weights are known to one skilled in the art, which may be used to create PEG-hGH conjugates or PEG-hGH agonist variant conjugates (for reviews see Roberts M. J. et al., Adv. Drug Del. Rev. 54:459-476, 2002), Harris J. M. et al., Drug Delivery Sytems 40:538-551, 2001) The amino acid residue may be any reactive one(s) having, for example, free amino, carboxyl, sulfhydryl (thiol), hydroxyl, guanidinyl, or imidizoyl groups, to which a terminal reactive group of an activated poly(ethylene glycol) may be bound. The amino acid residues having the free amino groups may include lysine residues and/or N-terminal amino acid residue, those having a free carboxyl group may include aspartic acid, glutamic acid and/or C-terminal amino acid residues, those having a free sulfhydryl (thiol) such as cysteine, those having a free hydroxyl such as serine or tyrosine, those having a free guanidinyl such as arginine, and those having a free imidizoyl such as histidine.

In another embodiment, oxime chemistries (Lemieux & Bertozzi Tib Tech 16:506-513, 1998) are used to target N-terminal serine residues.

The poly(ethylene glycol) used in the present invention is not restricted to any particular form or molecular weight range. The poly(ethylene glycol) molecular weight may between 500 and 100,000. Normally, a molecular weight of 500-60,000 is used and preferably of from 1,000-40,000. More preferable, the molecular weight is greater than 5,000 to about 40,000.

In another embodiment the poly(ethylene glycol) is a branched PEG having more than one PEG moiety attached. Preferred examples of branched PEGs are described in U.S. Pat. No. 5,932,462; U.S. Pat. No. 5,342,940; U.S. Pat. No. 5,643,575; U.S. Pat. No. 5,919,455; U.S. Pat. No. 6,113,906; U.S. Pat. No. 5,183,660; WO 02/09766; Kodera Y., Bioconjugate Chemistry 5:283-288 (1994); and Yamasaki et al., Agric. Biol. Chem., 52:2125-2127, 1998. In a preferred embodiment the molecular weight of each poly(ethylene glycol) of the branched PEG is 5,000-20,000.

Poly(alkylene oxide)s, notably poly(ethylene glycol)s, are bound to hGH or agonist variant thereof via a terminal reactive group, which may or may not leave a linking moiety (spacer) between the PEG and the protein. In order to form the hGH conjugates or agonist variant thereof of the present invention, polymers such as poly(alkylene oxide) are converted into activated forms, as such term is known to those of ordinary skill in the art. The reactive group, for example, is a terminal reactive group, which mediates a bond between chemical moieties on the protein, such as amino, carboxyl or thiol groups, and poly(ethylene glycol). Typically, one or both of the terminal polymer hydroxyl end-groups, (i.e. the alpha and omega terminal hydroxyl groups) are converted into reactive functional groups, which allows covalent conjugation. This process is frequently referred to as “activation” and the poly(ethylene glycol) product having the reactive group is hereinafter referred to as “an activated poly(ethylene glycol)”. Polymers containing both α and ε linking groups are referred to as “bis-activated poly(alkylene oxides)” and are referred to as “bifunctional”. Polymers containing the same reactive group on α and ε terminal hydroxyls are sometimes referred to as “homobifunctional” or “homobis-activated”. Polymers containing different reactive groups on α and ε terminal hydroxyls are sometimes referred to as “heterobifunctional” (see for example WO 01/26692) or “heterobis-activated”. Polymers containing a single reactive group are referred to as “mono-activated” polyalkylene oxides or “mono-functional”. Other substantially non-antigenic polymers are similarly “activated” or “functionalized”.

The activated polymers or reactive polymers are thus suitable for mediating a bond between chemical moieties on the protein, such as α- or ε-amino, carboxyl or thiol groups, and poly(ethylene glycol). Bis-activated polymers can react in this manner with two protein molecules or one protein molecule and a reactive small molecule in another embodiment to effectively form protein polymers or protein-small molecule conjugates through cross linkages.

Functional groups capable of reacting with either the amino terminal α-amino group or ε-amino groups of lysines found on the hGH or agonist variant thereof include: N-hydroxysuccinimidyl esters (U.S. Pat. No. 5,672,662); carbonate esters such as the p-nitrophenyl, or succinimidyl esters (U.S. Pat. No. 5,808,096, U.S. Pat. Nos. 5,650,234, 5,612,460, U.S. Pat. No. 5,324,844, U.S. Pat. No. 5,5,122,614); carbonyl imidazole; azlactones (U.S. Pat. No. 5,321,095, U.S. Pat. No. 5,567,422); cyclic imide thiones (U.S. Pat. No. 5,405,877, 5,349,001); dichlorotriazine (U.S. Pat. No. 5,147,537); imidates (U.S. Pat. No. 5,109,120) or thioimidates; acid chloride; isocyanates or isothiocyanates (Greenwald R. B., J. Org. Chem., 60:331-336, 1995); tresyl chloride (EP 714 402, EP 439 508); halogenformates (WO 96/40792), aldehyde (U.S. Pat. No. 4,002,531) or aldehyde hydrates (U.S. Pat. No. 5,990,237); and combination of carboxylic acid and activating agents such as N,N′-dicyclohexyl-cabodiimide (DCC), N-(dimethylaminopropyl)-N′-ethylcarbodiimide (EDC), diphenylphosphoryl azide (DPPA) or isobutylchloro-formate (Peptide Chemistry, A Practical Textbook, 2nd ed., Miklos Bodanszky, Springer-Verlarg, Berlin, 1993).

Functional groups capable of reacting with carboxylic acid groups, reactive carbonyl groups and oxidized carbohydrate moieties on hGH or agonist variant thereof include; primary amines; and hydrazine and hydrazide functional groups such as the acyl hydrazides, carbazates, semicarbamates, thiocarbazates, etc (WO 01/70685).

Mercapto groups, if available on the hGH or agonist variant thereof, can also be used as attachment sites for suitably activated polymers with reactive groups such as thiols; maleimides, sulfones, and phenyl glyoxals; see, for example, U.S. Pat. No. 5,093,531, the disclosure of which is hereby incorporated by reference. Other nucleophiles capable of reacting with an electrophilic center include, but are not limited to, for example, hydroxyl, amino, carboxyl, thiol, active methylene and the like.

Also included are polymers including lipophilic and hydrophilic moieties disclosed in U.S. Pat. No. 5,359,030 and U.S. Pat. No. 5,681,811; U.S. Pat. No. 5,438,040; and U.S. Pat. No. 5,359,030.

As well halogenated PEGs are disclosed on WO 98/32466 that can react with amino, thiol groups, and aromatic hydroxy groups, which directly covalently attach the PEG to the protein.

In one preferred embodiment of the invention secondary amine or amide linkages are formed using the N-terminal α-amino group or ε-amino groups of lysine of hGH or agonist variant thereof and the activated PEG. In another preferred aspect of the invention, a secondary amine linkage is formed between the N-terminal primary α- or ε-amino group of hGH or agonist variant thereof and single or branched chain PEG aldehyde by reduction with a suitable reducing agent such as NaCNBH₃, NaBH₄, Pyridine Borane etc. as described in Chamow et al., Bioconjugate Chem. 5: 133-140 (1994) and U.S. Pat. No. 5,824,784.

In a preferred embodiment at least 70%, preferably at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, and most preferably at least 98% of the poly(ethylene glycol) is on the amino terminal α-amino group.

In another preferred embodiment of the invention, polymers activated with amide-forming linkers such as succinimidyl esters, cyclic imide thiones, or the like are used to effect the linkage between the hGH or agonist variant thereof and polymer, see for example, U.S. Pat. No. 5,349,001; U.S. Pat. No. 5,405,877; and Greenwald, et al., Crit. Rev. Ther. Drug Carrier Syst. 17:101-161, 2000, which are incorporated herein by reference. One preferred activated poly(ethylene glycol), which may be bound to the free amino groups of hGH or agonist variant thereof includes single or branched chain N-hydroxysuccinylimide poly(ethylene glycol) may be prepared by activating succinic acid esters of poly(ethylene glycol) with N-hydroxysuccinylimide.

Other preferred embodiments of the invention include using other activated polymers to form covalent linkages of the polymer with the hGH or agonist variant thereof via ε-amino or other groups. For example, isocyanate or isothiocyanate forms of terminally activated polymers can be used to form urea or thiourea-based linkages with the lysine amino groups (Greenwald R. B., J. Org. Chem., 60:331-336, 1995).

In another preferred aspect of the invention, carbamate (urethane) linkages are formed with protein amino groups as described in U.S. Pat. Nos. 5,122,614, 5,324,844, and 5,612,640, which are hereby incorporated by reference. Examples include N-succinimidyl carbonate, para-nitrophenyl carbonate, and carbonyl imidazole activated polymers. In another preferred embodiment of this invention, a benzotriazole carbonate derivative of PEG is linked to amino groups on hGH or agonist variant thereof.

Another aspect of the invention represents a prodrug or sustained release form of hGH or agonist variant thereof, comprised of a water soluble polymer, such as poly(ethylene glycol), attached to an hGH or agonist variant thereof molecule by a functional linker that can predictably break down by enzymatic or pH directed hydrolysis to release free hGH or agonist variant thereof or other hGH or agonist variant thereof derivative. The prodrug can also be a “double prodrug” (Bundgaard in Advanced Drug Delivery Reviews 3:39-65, 1989) involving the use of a cascade latentiation. In such systems, the hydrolytic reaction involves an initial rate-limiting (slow) enzymatic or pH directed step and a second step involving a rapid non-enzymatic hydrolysis that occurs only after the first has taken place. Such a releasable polymer provides protein conjugates, which are impermanent and could act as a reservoir, that continually discharge hGH or agonist variant thereof. Such functional linkers are described in U.S. Pat. No. 5,614,549; U.S. Pat. No. 5,840,900; U.S. Pat. No. 5,880,131; U.S. Pat. No. 5,965,119; U.S. Pat. No. 5,965,565; U.S. Pat. No. 6,011,042; U.S. Pat. No. 6,153,655; U.S. Pat. No. 6,180,095 B1; U.S. Pat. No. 6,413,507; Greenwald R. B. et al., J. Med. Chem. 42;3657-3667, 1999; Lee, S. et al., Bioconjugate Chem 12:163-169, 2001; Garman A. J. et al., FEBS Lett. 223:361-365, 1987; Woghiren C. et al., Bioconjucate Chem. 4:314-318, 1993; Roberts M. J. et al., J. Pharm. Sci. 87;1440-1445, 1998; Zhao X., in Ninth Int. Symp. Recent Adv. Drug Delivery Syst. 199; Greenwald R. B. et al., J. Med. Chem. 43:475-487, 2000; and Greenwald R. B. Crit. Rev. Ther. Drug Carrier Syst. 17:101-161, 2000. Zalipsky et al., 28^{th} Int. Symp. On controlled Release of Bioactive Materials 1; 73-74,2001

Conjugation reactions, referred to as pegylation reactions, were historically carried out in solution with molar excess of polymer and without regard to where the polymer will attach to the protein. Such general techniques, however, have typically been proven inadequate for conjugating bioactive proteins to non-antigenic polymers while retaining sufficient bioactivity. One way to maintain the hGH or agonist variant thereof bioactivity is to substantially avoid the conjugation of those hGH or agonist variant thereof reactive groups associated with the receptor binding site(s) in the polymer coupling process. Another aspect of the present invention is to provide a process of conjugating poly(ethylene glycol) to hGH or agonist variant thereof maintaining high levels of retained activity.

The chemical modification through a covalent bond may be performed under any suitable condition generally adopted in a reaction of a biologically active substance with the activated poly(ethylene glycol). The conjugation reaction is carried out under relatively mild conditions to avoid inactivating the hGH or agonist variant thereof. Mild conditions include maintaining the pH of the reaction solution in the range of 3 to 10 and the reaction temperatures within the range of from about 0°-37° C. In the cases where the reactive amino acid residues in hGH or agonist variant thereof have free amino groups, the above modification is preferably carried out in a non-limiting list of suitable buffers (pH 3 to 10), including phosphate, MES, citrate, acetate, succinate or HEPES, for 1-48 hrs at 4°-37° C. In targeting N-terminal amino groups with reagents such as PEG aldehydes pH 4-8 is preferably maintained. The activated poly(ethylene glycol) may be used in about 0.05-100 times, preferably about 0.01-2.5 times, the molar amount of the number of free amino groups of hGH or agonist variant thereof. On the other hand, where reactive amino acid residues in hGH or agonist variant thereof have the free carboxyl groups, the above modification is preferably carried out in pH from about 3.5 to about 5.5, for example, the modification with poly(oxyethylenediamine) is carried out in the presence of carbodiimide (pH 3.5-5) for 1-24 hrs at 4°-37° C. The activated poly(ethylene glycol) may be used in 0.05-300 times the molar amount of the number of free carboxyl groups of hGH or agonist variant thereof.

In separate embodiments, the upper limit for the amount of polymer included in the conjugation reactions exceeds about 1:1 to the extent that it is possible to react the activated polymer and hGH or agonist variant thereof without forming a substantial amount of high molecular weight species, i.e. more than about 20% of the conjugates containing more than about one strand of polymer per molecule of hGH or agonist variant thereof. For example, it is contemplated in this aspect of the invention that ratios of up to about 6:1 can be employed to form significant amounts of the desired conjugates which can thereafter be isolated from any high molecular weight species.

In another aspect of this invention, bifunctionally activated PEG derivatives may be used to generate polymeric hGH or agonist variant thereof-PEG molecules in which multiple hGH or agonist variant thereof molecules are crosslinked via PEG. Although the reaction conditions described herein can result in significant amounts of unmodified hGH or agonist variant thereof, the unmodified hGH or agonist variant thereof can be readily recycled into future batches for additional conjugation reactions. The processes of the present invention generate surprisingly very little, i.e. less than about 30% and more preferably, less than about 10%, of high molecular weight species and species containing more than one polymer strand per hGH or agonist variant thereof. These reaction conditions are to be contrasted with those typically used for polymeric conjugation reactions wherein the activated polymer is present in several-fold molar excesses with respect to the target. In other aspects of the invention, the polymer is present in amounts of from about 0.1/amino group to about 50 equivalents per equivalent of hGH or agonist variant thereof. In other aspects of the invention, the polymer is present in amounts of from about 1 to about 10 equivalents per equivalent of hGH or agonist variant thereof.

The conjugation reactions of the present invention initially provide a reaction mixture or pool containing mono- and di-PEG-hGH conjugates, unreacted hGH, unreacted polymer, and usually less than about 20% high molecular weight species. The high molecular weight species include conjugates containing more than one polymer strand and/or polymerized PEG-hGH or agonist variant thereof species. After the unreacted species and high molecular weight species have been removed, compositions containing primarily mono- and di-polymer-hGH or agonist variant thereof conjugates are recovered. Given the fact that the conjugates for the most part include a single polymer strand, the conjugates are substantially homogeneous. These modified hGH or agonist variant thereof have at least about 0.1% of the in vitro biological activity associated with the native or unmodified hGH or agonist variant thereof as measured using standard FDC-P1 cell proliferation assays, (Clark et al. Journal of Biological Chemistry 271:21969-21977, 1996), receptor binding assay (U.S. Pat. No. 5,057,417), or hypophysectomized rat growth (Clark et al. Journal of Biological Chemistry 271:21969-21977, 1996). In preferred aspects of the invention, however, the modified hGH or agonist variant thereof have about 25% of the in vitro biological activity, more preferably, the modified hGH or agonist variant thereof have about 50% of the in vitro biological activity, more preferably, the modified hGH or agonist variant thereof have about 75% of the in vitro biological activity, and most preferably the modified hGH or agonist variant thereof have equivalent or improved in vitro biological activity.

The processes of the present invention preferably include rather limited ratios of polymer to hGH or agonist variant thereof. Thus, the hGH or agonist variant thereof conjugates have been found to be predominantly limited to species containing only one strand of polymer. Furthermore, the attachment of the polymer to the hGH or agonist variant thereof reactive groups is substantially less random than when higher molar excesses of polymer linker are used. The unmodified hGH or agonist variant thereof present in the reaction pool, after the conjugation reaction has been quenched, can be recycled into future reactions using ion exchange or size exclusion chromatography or similar separation techniques.

A poly(ethylene glycol)-modified hGH or agonist variant thereof, namely chemically modified protein according to the present invention, may be purified from a reaction mixture by conventional methods which are used for purification of proteins, such as dialysis, salting-out, ultrafiltration, ion-exchange chromatography, hydrophobic interaction chromatography (HIC), gel chromatography and electrophoresis. Ion-exchange chromatography is particularly effective in removing unreacted poly(ethylene glycol) and hGH or agonist variant thereof. In a further embodiment of the invention, the mono- and di-polymer-hGH or agonist variant thereof species are isolated from the reaction mixture to remove high molecular weight species, and unmodified hGH or agonist variant thereof. Separation is effected by placing the mixed species in a buffer solution containing from about 0.5-10 mg/mL of the hGH or agonist variant thereof-polymer conjugates. Suitable solutions have a pH from about 4 to about 8. The solutions preferably contain one or more buffer salts selected from KCl, NaCl, K₂HPO₄, KH₂PO₄, Na₂HPO₄, NaH₂PO₄, NaHCO₃, NaBO₄, CH₃CO₂H, and NaOH.

Depending upon the reaction buffer, the hGH or agonist variant thereof polymer conjugate solution may first have to undergo buffer exchange/ultrafiltration to remove any unreacted polymer. For example, the PEG-hGH or agonist variant thereof conjugate solution can be ultrafiltered across a low molecular weight cut-off (10,000 to 30,000 Dalton) membrane to remove most unwanted materials such as unreacted polymer, surfactants, if present, or the like.

The fractionation of the conjugates into a pool containing the desired species is preferably carried out using an ion exchange chromatography medium. Such media are capable of selectively binding PEG-hGH or agonist variant thereof conjugates via differences in charge, which vary in a somewhat predictable fashion. For example, the surface charge of hGH or agonist variant thereof is determined by the number of available charged groups on the surface of the protein. These charged groups typically serve as the point of potential attachment of poly(alkylene oxide) polymers. Therefore, hGH or agonist variant thereof conjugates will have a different charge from the other species to allow selective isolation.

Strongly polar anion or cation exchange resins such as quaternary amine or sulfopropyl resins, respectively, are used for the method of the present invention. Ion exchange resins are especially preferred. A non-limiting list of included commercially available cation exchange resins suitable for use with the present invention are SP-hitrap®, SP Sepharose HP® and SP Sepharose® fast flow. Other suitable cation exchange resins e.g. S and CM resins can also be used. A non-limiting list of anion exchange resins, including commercially available anion exchange resins, suitable for use with the present invention are Q-hitrap®, Q Sepharose HP®, and Q sepharose® fast flow. Other suitable anion exchange resins, e.g. DEAE resins, can also be used.

For example, the anion or cation exchange resin is preferably packed in a column and equilibrated by conventional means. A buffer having the same pH and osmolality as the polymer conjugated hGH or agonist variant thereof solution is used. The elution buffer preferably contains one or more salts selected from KCl, NaCl, K₂HPO₄, KH₂PO₄, Na₂HPO₄, NaH₂PO₄, NaHCO₃, NaBO₄, and (NH₄)₂CO₃. The conjugate-containing solution is then adsorbed onto the column with unreacted polymer and some high molecular weight species not being retained. At the completion of the loading, a gradient flow of an elution buffer with increasing salt concentrations is applied to the column to elute the desired fraction of polyalkylene oxide-conjugated hGH or agonist variant thereof. The eluted pooled fractions are preferably limited to uniform polymer conjugates after the cation or anion exchange separation step. Any unconjugated hGH or agonist variant thereof species can then be back washed from the column by conventional techniques. If desired, mono and multiply pegylated hGH or agonist variant thereof species can be further separated from each other via additional ion exchange chromatography or size exclusion chromatography.

Techniques utilizing multiple isocratic steps of increasing concentration of salt or pH can also be used. Multiple isocratic elution steps of increasing concentration will result in the sequential elution of di- and then mono-hGH or agonist variant thereof-polymer conjugates.

The temperature range for elution is between about 4° C. and about 25° C. Preferably, elution is carried out at a temperature of from about 4° C. to about 22° C. For example, the elution of the PEG-hGH or agonist variant thereof fraction is detected by UV absorbance at 280 nm. Fraction collection may be achieved through simple time elution profiles.

A surfactant can be used in the processes of conjugating the poly(ethylene glycol) polymer with the hGH or agonist variant thereof moiety. Suitable surfactants include ionic-type agents such as sodium dodecyl sulfate (SDS). Other ionic surfactants such as lithium dodecyl sulfate, quaternary ammonium compounds, taurocholic acid, caprylic acid, decane sulfonic acid, etc. can also be used. Non-ionic surfactants can also be used. For example, materials such as poly(oxyethylene) sorbitans (Tweens), poly(oxyethylene) ethers (Tritons) can be used. See also Neugebauer, A Guide to the Properties and Uses of Detergents in Biology and Biochemistry (1992) Calbiochem Corp. The only limitations on the surfactants used in the processes of the invention are that they are used under conditions and at concentrations that do not cause substantial irreversible denaturation of the hGH or agonist variant thereof and do not completely inhibit polymer conjugation. The surfactants are present in the reaction mixtures in amounts from about 0.01-0.5%; preferably from 0.05-0.5%; and most preferably from about 0.075-0.25%. Mixtures of the surfactants are also contemplated.

It is thought that the surfactants provide a temporary, reversible protecting system during the polymer conjugation process. Surfactants have been shown to be effective in selectively discouraging polymer aggregates while allowing lysine-based or amino terminal-based conjugation to proceed.

The present poly(ethylene glycol)-modified hGH or agonist variant thereof has a more enduring pharmacological effect, which may be possibly attributed to its prolonged half-life in vivo.

Furthermore, the present poly(ethylene glycol)-modified hGH or agonist variant thereof may be useful for the treatment of hypo pituitary dwarfism (GHD), Adult Growth Hormone Deficiency, Turner's syndrome, growth failure in children who were born short for gestational age (SGA), Prader-Willi syndrome (PWS), chronic renal insufficiency (CRI), Aids wasting, and Aging.

The present poly(ethylene glycol)-modified hGH or agonist variant thereof may be formulated into pharmaceuticals containing also a pharmaceutically acceptable diluent, an agent for preparing an isotonic solution, a pH-conditioner and the like in order to administer them into a patient.

The above pharmaceuticals may be administered subcutaneously, intramuscularly, intravenously, pulmonary, intradermally, or orally, depending on a purpose of treatment. A dose may be also based on the kind and condition of the disorder of a patient to be treated, being normally between 0.1 mg and 5 mg by injection and between 0.1 mg and 50 mg in an oral administration for an adult

The polymeric substances included are also preferably water-soluble at room temperature. A non-limiting list of such polymers include poly(alkylene oxide) homopolymers such as poly(ethylene glycol) or poly(propylene glycols), poly(oxyethylenated polyols), copolymers thereof and block copolymers thereof, provided that the water solubility of the block copolymers is maintained.

As an alternative to PEG-based polymers, effectively non-antigenic materials such as dextran, poly(vinyl pyrrolidones), poly(acrylamides), poly(vinyl alcohols), carbohydrate-based polymers, and the like can be used. Indeed, the activation of α- and ω-terminal groups of these polymeric substances can be effected in fashions similar to that used to convert poly(alkylene oxides) and thus will be apparent to those of ordinary skill. Those of ordinary skill in the art will realize that the foregoing list is merely illustrative and that all polymer materials having the qualities described herein are contemplated. For purposes of the present invention, “effectively non-antigenic” means all materials understood in the art as being nontoxic and not eliciting an appreciable immunogenic response in mammals.

Definitions

The following is a list of abbreviations and the corresponding meanings as used interchangeably herein:

| | |
|---|---|
| g | gram(s) |
| mg | milligram(s) |
| ml or mL | milliliter(s) |
| RT | room temperature |
| PEG | poly (ethylene glycol) |

The complete content of all publications, patents, and patent applications cited in this disclosure are herein incorporated by reference as if each individual publication, patent, or patent application were specifically and individually indicated to be incorporated by reference.

Although the foregoing invention has been described in some detail by way of illustration and example for the purposes of clarity of understanding, it will be readily apparent to one skilled in the art in light of the teachings of this invention that changes and modifications can be made without departing from the spirit and scope of the present invention. The following examples are provided for exemplification purposes only and are not intended to limit the scope of the invention, which has been described in broad terms above.

In the following examples, the hGH is that of SEQ ID NO: 1. It is understood that other members of the hGH or agonist variant thereof family of polypeptides could also be pegylated in a similar manner as exemplified in the subsequent examples.

All references, patents or applications cited herein are incorporated by reference in their entirety as if written herein.

The present invention will be further illustrated by referring to the following examples, which however, are not to be construed as limiting the scope of the present invention.

### EXAMPLES

### Example 1

Straight Chain 20,000 MW PEG-ALD hGH

mPEG-O—CH₂CH₂CH₂—NH-hGH

This example demonstrates a method for generation of substantially homogeneous preparations of N-terminally monopegylated hGH by reductive alkylation. Methoxy-linear PEG-propionaldehyde reagent of approximately 20,000 MW (Shearwater Corp.) was selectively coupled via reductive amination to the N-terminus of hGH by taking advantage of the difference in the relative pKₐ value of the primary amine at the N-terminus versus pKₐ values of primary amines at the ε-amino position of lysine residues. hGH protein dissolved at 10 mg/mL in 25 mM MES (Sigma Chemical, St. Louis, Mo.) pH 6.0, 25 mM Hepes (Sigma Chemical, St. Louis, Mo.) pH 7.0, or in 10 mM Sodium Acetate (Sigma Chemical, St. Louis, Mo.) pH 4.5, was reacted with Methoxy-PEG-propionaldehyde, M-PEG-ALD, (Shearwater Corp., Huntsville, Ala.) by addition of M-PEG-ALD to yield a relative PEG:hGH molar ratio of 0.1:0.7 per amine (optionally 8% acetonitrile may also be added). Reactions were catalyzed by addition of stock 1M NaCNBH₄ (Sigma Chemical, St. Louis, Mo.), dissolved in H₂O, to a final concentration of 10-50 mM. Reactions were carried out in the dark at 4° C. to RT for 18-24 hours. Reactions were stopped by addition of 1 M Tris (Sigma Chemical, St. Louis, Mo.) ˜pH 7.6 to a final Tris concentration of 50 mM or diluted into appropriate buffer for immediate purification.

### Example 2

Straight Chain 30,000 MW PEG-ALD hGH

Methoxy-linear 30,000 MW PEG-propionaldehyde reagent (Shearwater Corp.) was coupled to the N-terminus of hGH using the procedure described for Example 1.

### Example 3

Straight Chain 5,000 MW PEG-ALD hGH

Methoxy-linear 5,000 MW PEG-propionaldehyde reagent (Fluka) was coupled to the N-terminus of hGH using the procedure described for Example 1.

### Example 4

Branched Chain 40,000 MW PEG-ALD hGH

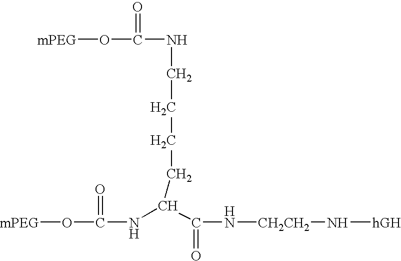

Methoxy-branched 40,000 MW PEG-aldehyde (PEG2-ALD) reagent (Shearwater Corp.) was coupled to the N-terminus of hGH using the procedure described for Example 1.

### Example 5

Branched Chain 20,000 MW PEG-ALD hGH

Methoxy-branched 20,000 MW PEG-aldehyde (PEG2-ALD) reagent (Shearwater Corp.) was coupled to the N-terminus of hGH using the procedure described for Example 1 using PEG to hGH molar ratios of 0.1-0.5 per amine.

### Example 6

Straight Chain 30,000 MW SPA-PEG hGH

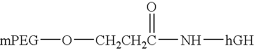

This example demonstrates a method for generation of substantially homogeneous preparations of mono-pegylated hGH using N-hydroxysuccinimidyl (NHS) active esters. hGH protein stock solution was dissolved at 10 mg/mL in 0.25 M HEPES buffer, pH 7.2 (optionally 8% acetonitrile may also be added). The solution was then reacted with Methoxy-PEG-succinimidyl propionate (SPA-PEG) by addition of SPA-PEG to yield a relative PEG:hGH molar ratio of 0.1-0.65 per amine. Reactions were carried out at 4° C. to RT for from 5 minutes to 1 hour. Reactions were stopped by lowering the pH to 4.0 with 0.1 N acetic acid or by adding a 5× molar excess of Tris HCl.

### Example 7

Straight Chain 20,000 MW SPA-PEG hGH

Straight chain 20,000 MW SPA-PEG reagent (Shearwater Corp.) was coupled to the N-terminus of hGH using the procedure described for Example 6.

### Example 8

Straight Chain 3,400 MW Biotin-SPA-PEG-hGH

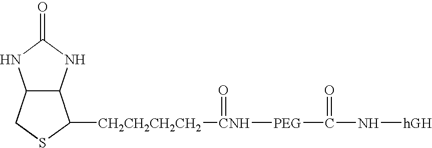

3,400 MW Biotin-PEG-CO₂—NHS reagent (Shearwater Corp.) is coupled to hGH using the procedure described for Example 6.

### Example 9

Branched 10,000 MW NHS-PEG-hGH

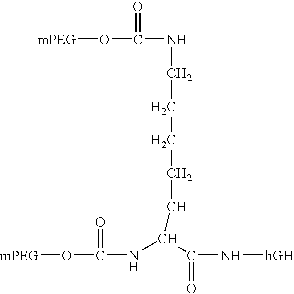

10,000 MW branched PEG2-NHS (Shearwater Corp.) is coupled to hGH using the procedure described for Example 6.

### Example 10

Branched 20,000 MW NHS-PEG-hGH

20,000 MW branched PEG2-NHS (Shearwater Corp.) is coupled to hGH using the procedure described for Example 6.

### Example 11

Branched 40,000 MW NHS-PEG-hGH

40,000 MW branched PEG2-NHS (Shearwater Corp.) was coupled to hGH using the procedure described for Example 6.

### Example 12

Straight Chain 20,000 MW PEG-BTC-hGH

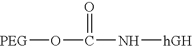

20,000 MW PEG-BTC (Shearwater Corp.) is coupled to hGH using the procedure described for Example 6. This example demonstrates a method for generation of substantially homogeneous preparations of pegylated hGH using benzotriazole carbonate derivatives of PEG.

### Example 13

Straight Chain 5,000 MW PEG-SS-hGH

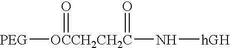

5,000 MW succinimidyl succinate-PEG (SS-PEG) (Shearwater Corp.) is coupled to hGH using the procedure described for Example 6. This example demonstrates a method for generation of substantially homogeneous preparations of pegylated hGH using a hydrolyzable linkage.

### Example 14

Straight Chain 20,000 MW PEG-CM-HBA-hGH

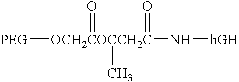

20,000 MW carboxymethyl hydroxybutyric acid-PEG (CM-HBA-PEG)(Shearwater Corp.) was coupled to hGH using the procedure described for Example 6. This example demonstrates a method for generation of substantially homogeneous preparations of pegylated hGH using a hydrolyzable linkage.

### Example 15

Straight Chain 2-4×5,000 MW PEG-CM-HBA-hGH

5,000 MW PEG-CM-HBA (Shearwater Corp.) was coupled to hGH using the procedure described for Example 13.

### Example 16

Straight Chain 20,000 MW HZ-PEG hGH

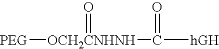

This example demonstrates a method for generation of substantially homogeneous preparations of pegylated hGH using 20,000 MW methoxy-PEG-hydrazide, HZ-PEG (Shearwater Corp.). hGH protein stock solution was dissolved at 10 mg/mL in 10 mM MES, pH 4.0. The solution was then reacted with HZ-PEG by addition of solid to yield a relative PEG:hGH molar ratio of 0.1-5.0 per carboxyl group. Reactions were catalyzed with carbodiimide (EDC, EOAC, EDEC) at a final concentration of 2 mM to 4 mM. Reactions were carried out at 4° C. for 2 hours to overnight or room temperature from 10 minutes to overnight. Reactions were stopped by Removing the unconjugated PEG and the carbodiimide by purification on cation exchange.

### Examples 17

Multi-Pegylated Species

Modified hGHs having two or more PEGs (multi-pegylated) attached were also obtained from Examples 1 and 4 and were separated from the mono-pegylated species using anion exchange chromatography. Modified hGHs having two or more PEGs (multi-pegylated) attached are also separated from mono-PEGylated species using cation exchange chromatography.

Modified hGHs having two or more PEGs (multi-pegylated) attached are also obtained in examples 2,3,5-13 and are purified in similar fashion to examples 1 and 4.

### Example 18

Purification of Pegylated hGH

Pegylated hGH species were purified from the reaction mixture to >95% (SEC analysis) using a single ion exchange chromatography step.

Anion Exchange Chromatography

The PEG hGH species were purified from the reaction mixture to >95% (SEC analysis) using a single anion exchange chromatography step. Mono-pegylated hGH was purified from unmodified hGH and multi-pegylated hGH species using anion exchange chromatography. A typical 20K aldehyde hGH reaction mixture (5-100 mg protein), as described above, was purified on a Q-Sepharose Hitrap column (1 or 5 mL)(Amersham Pharmacia Biotech, Piscataway, N.J.) or Q-Sepharose fast flow column (26/20, 70 mL bed volume)(Amersham Pharmacia Biotech, Piscataway, N.J.) equilibrated in 25 mM HEPES, pH 7.3 (Buffer A). The reaction mixture was diluted 5-10× with buffer A and loaded onto the column at a flow rate of 2.5 mL/min. The column was washed with 8 column volumes of buffer A. Subsequently, the various hGH species were eluted from the column in 80-100 column volumes of Buffer A and a linear NaCl gradient of 0-100 mM. The eluant was monitored by absorbance at 280 nm (A₂₈₀) and 5 mL fractions were collected. Fractions were pooled as to extent of pegylation, e.g., mono, di, tri etc. (as assessed in example 15). The pool was then concentrated to 0.5-5 mg/mL in a Centriprep YM10 concentrator (Amicon, Technology Corporation, Northborough, Mass.). Protein concentration of pool was determined by A₂₈₀ using an extinction coefficient of 0.78. Total yield of purified mono 20 K PEG-aldehyde hGH from this process was 25-30%.

Cation Exchange Chromatography

Cation exchange chromatography is carried out on an SP Sepharose high performance column (Pharmacia XK 26/20, 70 ml bed volume) equilibrated in 10 mM sodium acetate pH 4.0 (Buffer B). The reaction mixture is diluted 10× with buffer B and loaded onto the column at a flow rate of 5 mL/min. Next the column is washed with 5 column volumes of buffer B, followed by 5 column volumes of 12% buffer C (10 mM acetate pH 4.5, 1 M NaCl). Subsequently, the PEG-hGH species is eluted from the column with a linear gradient of 12 to 27% buffer C in 20 column volumes. The eluant is monitored at 280 nm and 10 mL fractions are collected. Fractions are pooled according to extent of pegylation (mono, di, tri etc.), exchanged into 10 mM acetate pH 4.5 buffer and concentrated to 1-5 mg/mL in a stirred cell fitted with an Amicon YM10 membrane. Protein concentration of pool is determined by A280 nm using an extinction coefficient of 0.78. Total yield of monopegylated hGH from this process is 10 to 50%.

### Example 19

Biochemical Characterization

The purified pegylated hGH pools were characterized by reducing and non-reducing SDS-PAGE, non-denaturing and denaturing Size Exclusion Chromatography, analytical Anion Exchange Chromatography, N-terminal Sequencing, Hydrophobic Interaction Chromatography, and Reversed Phase HPLC.

Size Exclusion High Performance Liquid Chromatography (SEC-HPLC)

Non-Denaturing SEC-HPLC

The reaction of Methoxy-PEG of various attachment chemistries, sizes, linkers, and geometries with hGH, anion exchange purification pools and final purified products were assessed using non-denaturing SEC-HPLC. Analytical non-denaturing SEC-HPLC was carried out using a Tosohaas G4000PWXL column, 7.8 mm×30 cm, (Tosohaas Amersham Bioscience, Piscataway, N.J.) or Superdex 200 (Amersham Bioscience, Piscataway, N.J.) in 20 mM Phosphate pH 7.2, 150 mM NaCl at a flow rate of 0.5 mL/minute. PEGylation greatly increases the hydrodynamic volume of the protein resulting in a shift to an earlier retention time. New species were observed in the PEG aldehyde hGH reaction mixtures along with unmodified hGH. These PEGylated and non-PEGylated species were separated on Q-Sepharose chromatography, and the resultant purified mono PEG-Aldehyde hGH species were subsequently shown to elute as a single peak on non-denaturing SEC (>95% purity). The Q-Sepharose chromatography step effectively removed free PEG, hGH, and multi PEGylated hGH species from the mono-Pegylated hGH. Non-denaturing SEC-HPLC demonstrated that the effective size of the various PEGylated-hGH was much greater than their respective theoretical molecular weights (Table 1).

TABLE 1
Size Exclusion Chromatography (SEC)

| | MW | |
|---|---|---|
| | (Theoretical) | Size (SEC) |
| hGH | 22,000 | 21,000 |
| 4-6 × 5 K PEG-SPA GH | 47,000 | 128,000 |
| 2-4 × 5 K PEG-CMHBA (NES) GH | 37,000 | 71,000 |
| 20 K PEG-ALD OH | 42,000 | 120,000 |
| 20 K Branched PEG-ALD GH | 42,000 | 114,000 |
| 20 K PEG-CMHBA (NHS) GH | 42,000 | 115,000 |
| 20 k PEG-Hydrazide GH | 42,000 | 125,000 |
| 2 × 20 K PEG-ALD GH | 62,000 | 250,000 |
| 30 K PEG-ALD GH | 52,000 | 231,000 |
| 30 K PEG-SPA GH | 52,000 | 183,000 |
| 2 × 30 K PEG-SPA GH | 82,000 | 569,000 |
| 40 K Branched PEG-ALD GH | 62,000 | 330,000 |
| 40 K Branched PEG-NHS GH | 62,000 | 253,000 |

Denaturing SEC-HPLC

The reaction of the various Methoxy-PEGs with hGH, anion exchange purification, and final purified products were assessed using denaturing SEC-HPLC. Analytical denaturing SEC-HPLC was carried out using a Tosohaas 3000SWXL column 7.8 mm×30 cm (Tosohaas Pharmacia Biotech, Piscataway, N.J.) in 100 mM Phosphate pH 6.8, 0.1% SDS at a flow rate of 0.8 mL/minute. PEGylation greatly increases the hydrodynamic volume of the protein resulting in a shift to an earlier retention time. New species were observed in the 20K PEG aldehyde hGH reaction mixture along with unmodified hGH. These PEGylated and non-PEGylated species were separated on Q-Sepharose chromatography, and the resultant purified mono 20K PEG-Aldehyde hGH was subsequently shown to elute as a single peak on denaturing SEC (>95% purity). The Q-Sepharose chromatography step effectively removed free PEG, hGH, and multi PEGylated hGH species from the mono-Pegylated hGH.

SDS PAGE/PVDF Transfer

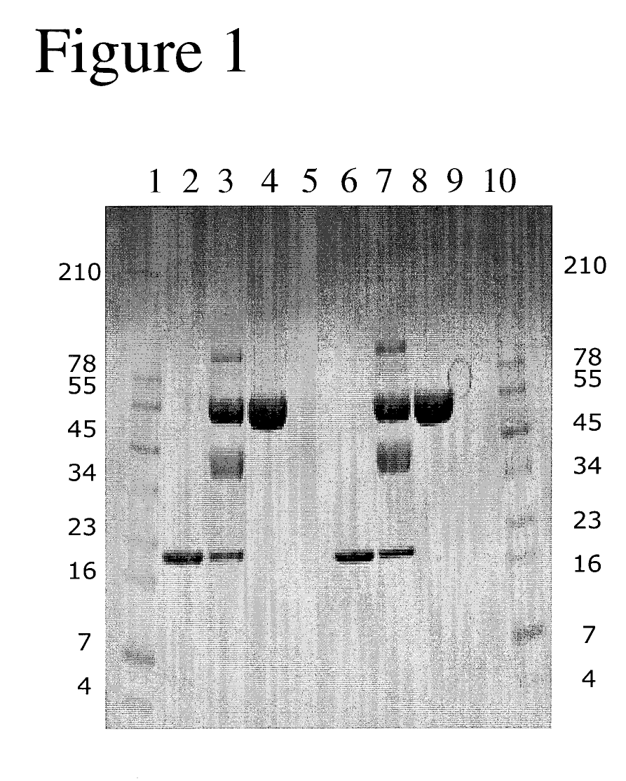

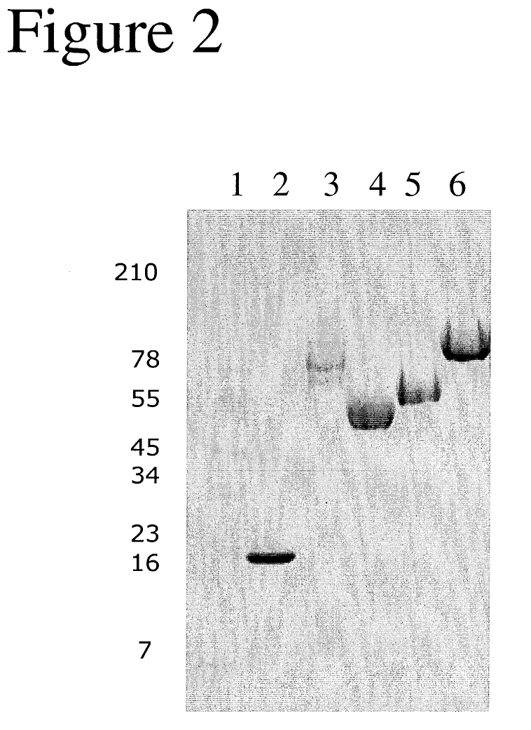

SDS-PAGE was used to assess the reaction of the various PEG reagents with hGH and the purified final products. Examples of this technique are shown with mono 20K linear and branched 20K and 40K PEG aldehyde, and 4×6 5K SPA PEG. (FIGS. 1 & 2). SDS-PAGE was carried out on 1 mm thick 10-20% Tris tricine gels (Invitrogen, Carlsbad, Calif.) under reducing and non-reducing conditions and stained using a Novex Colloidal Coomassie™ G-250 staining kit (Invitrogen, Carlsbad, Calif.). Purified mono PEG-aldehyde hGH species migrate as one major band on SDS-PAGE. Bands were blotted onto PVDF membrane for subsequent N-terminal sequence identification.

Analytical Anion Exchange HPLC

Analytical anion exchange HPLC was used to assess the reaction of various mPEGs with hGH, anion exchange purification fractions and final purified products. Analytical anion exchange HPLC was carried out using a Tosohaas Q5PW or DEAE-PW anion exchange column, 7.5 mm×75 mm (Tosohaas Pharmacia Biotech, Piscataway, N.J.) in 50 mM Tris ph 8.6 at a flow rate of 1 mL/min. Samples were eluted with a linear gradient of 5-200 mM NaCl.

Reversed Phase HPLC (RP-HPLC)

PEG-GH reaction mixtures and purified PEGylated products were analyzed by RP HPLC to elucidate hGH species, mono and multiply PEGylated hGH species, and, to monitor oxidized hGH forms, as well as, PEG hGH isoforms having a single PEG linked at different sites (e.g. N-terminus vs Lysine ε-amino groups). RP-HPLC was carried out utilizing a Zorbax SB-CN 150 or 250 mm×4.6 mm (3.5 mm or 5 mm) reversed phase HPLC column. Experiments were conducted at ambient temperature on a typical load of 10 mg of protein per sample. Buffer A is 0.1% triflouroacetic acid in water; Buffer B is 0.1% trifluoroacetic acid in acetonitrile. The gradient, which results in a ½% increase in B per minute, is as follows:

| Step | Time | Flow | % A | % B | Step |
|---|---|---|---|---|---|
| 0 | 0 | 1 | 60 | 40 | 0 |
| 1 | 3 | 1 | 60 | 40 | 0 |
| 2 | 20 | 1 | 50 | 50 | 1 |
| 3 | 2 | 1 | 60 | 40 | 1 |
| 4 | 6 | 1 | 60 | 40 | 0 |

N-Terminal Sequence and Peptide Mapping

Automated Edman degradation chemistry was used to determine the NH2-terminal protein sequence. An Applied Biosystems Model 494 Procise sequencer (Perkin Elmer, Wellesley, Mass.) was employed for the degradation. The respective PTH-AA derivatives were identified by RP-HPLC analysis in an on-line fashion employing an Applied Biosystems Model 140C PTH analyzer fitted with a Perkin Elmer/Brownlee 2.1 mm i.d. PTH-C18 column. 20K linear and 20 and 40K branched PEG-ALD hGH Protein bands transferred to PVDF membranes or solutions of purified 20K linear and branched 20 and 40K PEG-ALD hGH were sequenced. Purified 20K linear PEG-hGH yielded a major signal (approximately 88% yield) was observed that had the expected sequence for hGH except for the absence of the N-terminal amino acid. This result is as expected for a protein N-terminally PEGylated via the aldehyde chemistry. The residue of the first cycle is unrecoverable due to the attached PEG moiety. A minor signal (approximately 12% yield) had the correct N-terminal amino acid sequence. Considering that the peak collected off the RP-HPLC is 100% PEGylated, these data suggest that approximately 88% of the PEG modification is at the N-terminus with remainder apparently linked to one of several possible lysine residues.

Tryptic digests were performed at a concentration of 1 mg/mL and, typically, 50 ug of material is used per digest. Trypsin was added such that the trypsin to PEG-hGH ratio was 1:30 (w/w). Tris buffer was present at 30 mM, pH 7.5. Samples were incubated at room temperature for 16±0.5 hours. Reactions were quenched by the addition of 50 μL of 1N HCl per mL of digestion solution. Samples were diluted, prior to placing the samples in the autosampler, to a final concentration of 0.25 mg/ml in 6.25% acetonitrile. Acetonitrile is added first (to 19.8% acetonitrile), mixed gently, and then water is added to final volume (four times the starting volume). Extra digestion solution may be removed and stored for up to 1 week at −20° C.

A Waters Alliance 2695 HPLC system was used for analysis, but other systems should produce similar results. The column used was an Astec C-4 polymeric 25 cm×4.6 mm column with 5 μm particles. Experiments were conducted at ambient temperature on a typical load of 50 μg of protein per sample. Buffer A is 0.1% trifluoroacetic acid in water; buffer B is 0.085% trifluoroacetic acid in acetonitrile. The gradient is as follows:

| Time | A % | B % | C % | D % | Flow | Curve |
|---|---|---|---|---|---|---|
| 0.00 | 0.0 | 0.0 | 100.0 | 0.0 | 1.000 | 1 |
| 90.00 | 0.0 | 0.0 | 55.0 | 45.0 | 1.000 | 6 |
| 90.10 | 0.0 | 0.0 | 0.0 | 100.0 | 1.000 | 6 |
| 91.00 | 0.0 | 0.0 | 0.0 | 100.0 | 1.000 | 6 |
| 91.10 | 0.0 | 0.0 | 100.0 | 0.0 | 1.000 | 6 |
| 95.00 | 0.0 | 0.0 | 100.0 | 0.0 | 1.000 | 6 |

The column is heated to 40° C. using a heat jacket. Peaks were detected using a Waters 996 PDA detector collecting data between 210 and 300 nm. The extracted chromatogram at 214 nm was used for sample analysis to determine the extent of n-terminal Pegylation (loss of T-1 fragment) as shown in Table 2.

TABLE 2

| | | | % T-1 Present |
|---|---|---|---|
| | % T-1 | % T-1 | compared to |
| Sample | present | Lost | control |
| Aldehyde | | | |
| 5 K ALD | 2.0 | 98.0 | 7.4 |
| 20 K | 0.0 | 100.0 | 0.0 |
| 2 × 20 K | 0.0 | 100.0 | 0.0 |
| 30 K | 1.3 | 98.7 | 4.5 |
| 40 K | 1.9 | 98.1 | 6.8 |
| Branched | | | |
| NHS | | | |
| 4-6 × 5 SPA | 1.3 | 98.7 | 4.7 |
| 2-4 × 5 CM | 0.0 | 100.0 | 0.0 |
| 20 K CM | 23.1 | 76.9 | 84.1 |
| 30 K | 18.2 | 81.8 | 63.9 |
| 2 × 30 K | 5.7 | 94.3 | 19.9 |
| 40 K | 20.9 | 79.1 | 73.5 |
| branched | | | |

### Example 20

Pharmacodynainic Studies

Efficacy Studies in Hypophysectomized Rats

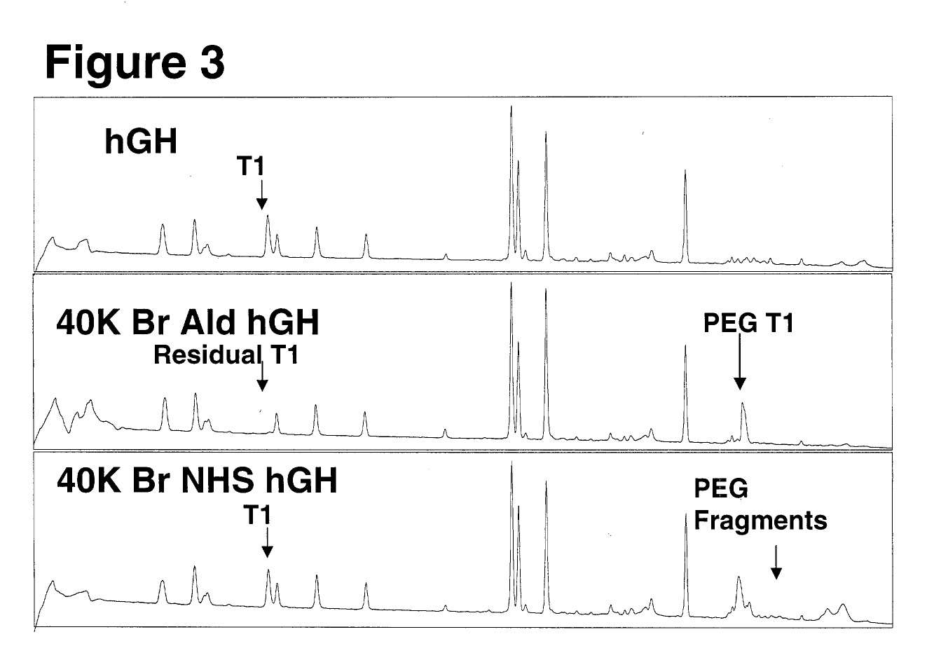

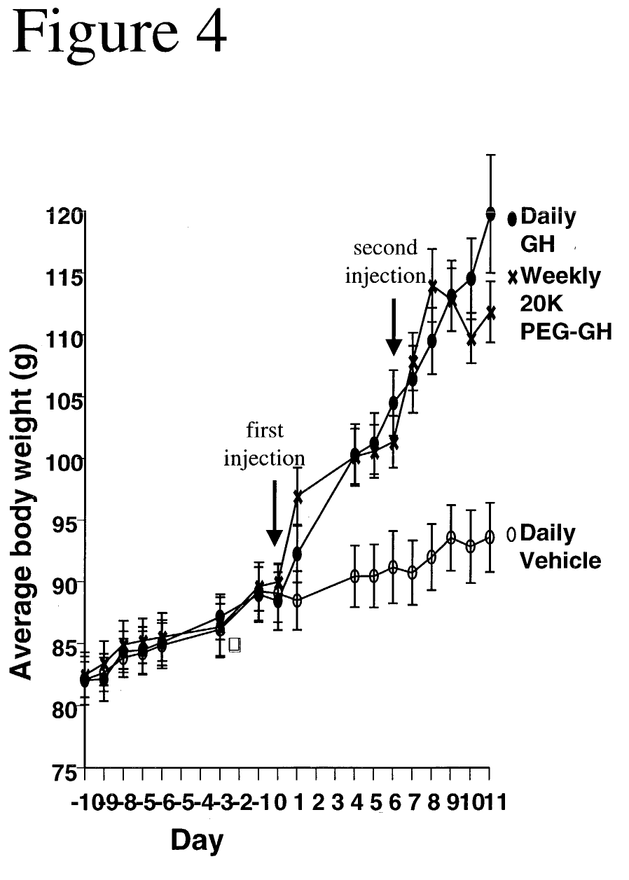

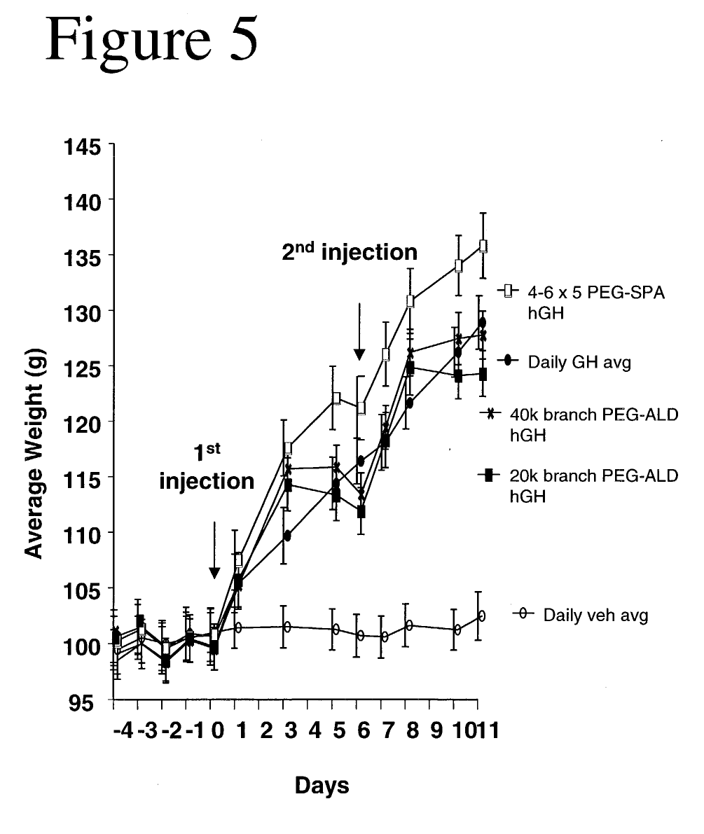

Female Sprague Dawley rats, hypophysectomized at Harlan Labs, were prescreened for growth rate for a period of 7 to 10 days. Subsequently, growth studies were carried out for 11 days. Rats were divided into groups of six to eight. Group 1 consisted of rats given either daily or day 0 and day 6 subcutaneous dose(s) of vehicle. Group 2 were given daily subcutaneous doses of GH (0.3 mg/kg/dose). Group 3 were given subcutaneous doses of GH on day 0 and day 6 (1.8 mg/kg/dose). Group 4 were given subcutaneous doses of PEG-GHs on day 0,6 (1.8 mg/kg/dose). Hypophysectomized rats were monitored for weight gain by weighing at least every other day during the study. Weight gains (average+/−SEM) for 20K PEG-ALD hGH, 20K and 40K branched PEG-ALD hGH, and 4-6×5 PEG-SPA hGH dosed once a week were similar to those for daily dosing of hGH (FIGS. 3 & 4) Table 3 summarizes total weight gain (average+/−SEM) at day 11 for once per week dosing of various Pegylated hGH molecules relative to daily dosing of hGH.

TABLE 3
Murine weight gain

| | | Daily | |
|---|---|---|---|
| | | weight gain | % weight |
| | | in | gain |
| | Single | grams/day | relative |
| | Weekly | (d0-d11) | to daily |
| | Dose | (Avg. ± | hGH gain |
| Compound | (mpk) | SEM) | (Avg.) |
| hGH (un-pegylated) | 1.8 | 0.97 ± 0.12 | 39% |
| 5 K Linear PEG-ALD GH | 1.8 | 0.96 ± 0.27 | 36% |
| 20 K Linear PEG-ALD | 1.8 | 1.99 ± 0.13 | 73% |
| GH | | 1.43 ± 0.08, | |
| | | 1.7 ± 0.10 | |
| 20 K Linear CM-HBA | 1.8 | 2.36 ± 0.11 | 99% |
| PEG GH | | | |
| 20 K Linear PEG-HYD | 1.8 | 2.62 ± 0.22 | 99% |
| GH | | | |
| 20 K Branched PEG-ALD | 1.8 | 2.24 ± 0.07 | 87% |
| GH | | | |
| 30 K Linear PEG-ALD | 1.8 | 2.11 ± 0.06; | 94% |
| GH | | 1.85 ± 0.14 | |
| 30 K Linear PEG-SPA | 1.8 | 2.6 +/− 0.1 | 117% |
| GH | | | |
| 40 K Branched PEG-ALD | 1.8 | 2.57 ± 0.08 | 100% |
| GH | | | |
| 40 K Branched PEG-NHS | 1.8 | 2.53 ± 0.09 | 121% |
| GH | | | |
| 2 × 20 K PEG-ALD GH | 1.8 | 2.66 ± 0.10 | 128% |
| 4-6 × 5 K SPA-PEG GH | 1.8 | 3.18 ± 0.10 | 124% |
| 2-4 × 5 K CM-HBA-PEG GH | 1.8 | 3.54 ± 0.15 | 134% |
| 2 × 30 K Linear PEG- | 1.8 | 3.1 ± 0.1 | 134% |
| SPA GH | | | |

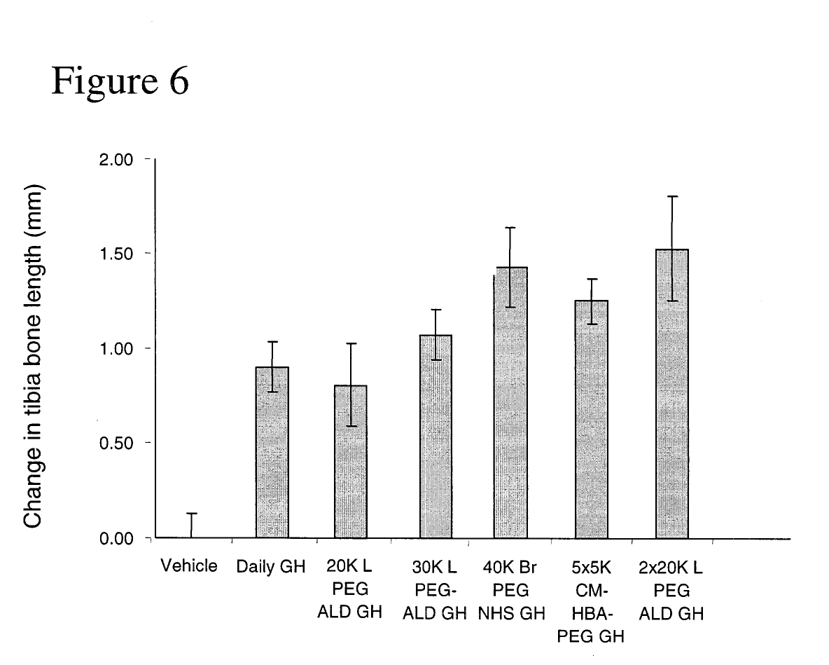

Upon completion of each growth study, animals were sacrificed and bone (tibia) lengths were analyzed. FIG. 6 shows the change in tibial bone length (average+/−SEM) at day 11 in response to various PEG-GH conjugates dosed on day 0 and day 6 or hGH dosed daily.

IGF-1 Levels in Hypophysectomized Rats

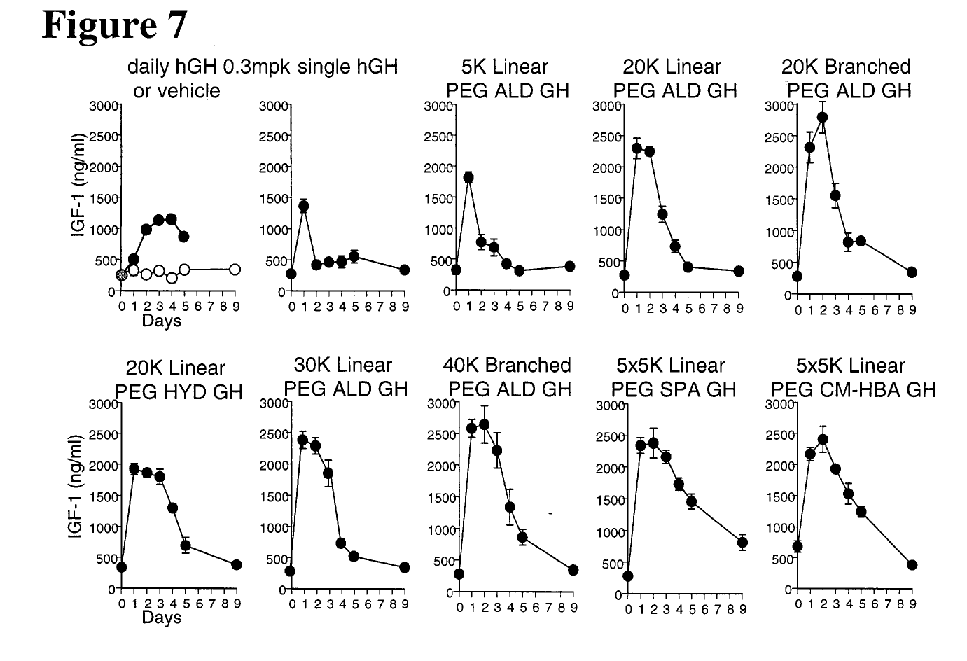

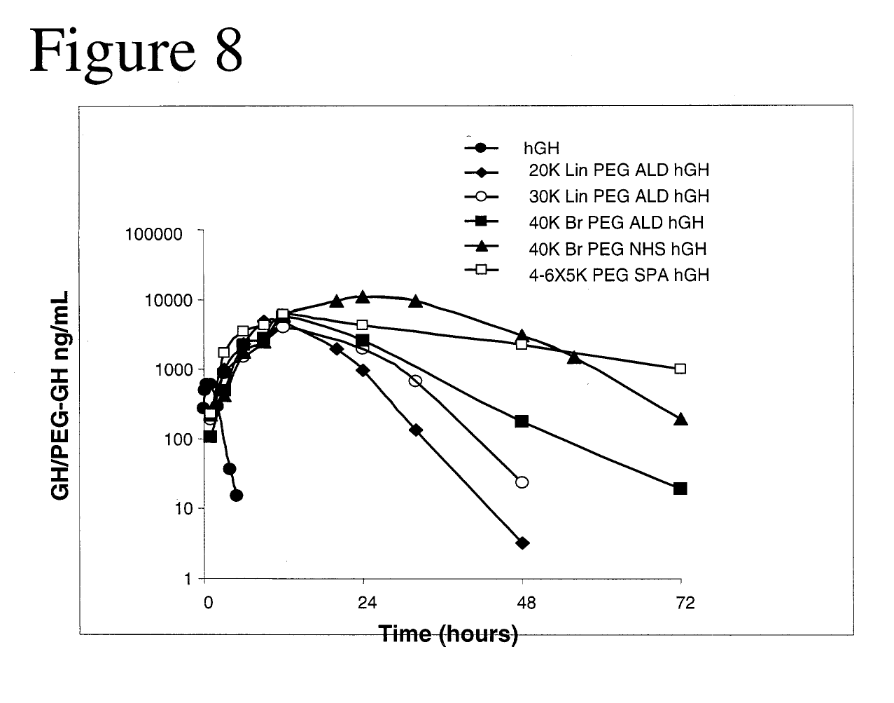

Experiments were carried out as above for the weight gain studies, however blood samples were taken at day 0, 1, 2, 3, 4, 5 and upon sacrifice of the animals at day 9. IGF-1 levels were determined by ELISA. FIG. 7 compares increases in serum IGF-1 levels (average+/−SEM) in hypophysectomized rats following either daily dosing of hGH or single dose of hGH or day 0, day 6 dosing of Pegylated hGH.

Pharmacokinetic Studies

Pharmacokinetic studies were conducted in normal, Sprague-Dawley male rats, mice, and cynomolgus monkeys. Injections were made, either as a single subcutaneous bolus of 1.8 mg/kg or as a single iv dose at 1.0 mg/kg GH or PEG-GH in rats and mice, using six rats and up to 60 mice per group. In cynomolgus monkeys a 0.18 mg/kg GH or PEG-GH dose was used for both single subcutaneous bolus and iv, using 2-4 monkeys per group. Blood samples were taken over one to five days as appropriate for assessment of relevant PK parameters (Table 4). (t_{1/2})=Terminal half life, (Cl)=clearance, (Tmax)=time to maximum concentration, Vss=volume distribution (apparent) at steady state, and (Cmax)=maximum concentration GH and PEG-GH blood levels were monitored at each sampling using immuno-assay.

hGH Immunoassay

hGH and pegylated hGH protein concentration levels in rat, mouse, and cynomolgus monkey plasma were determined using the hGH AutoDELFIA kit fluorescence immunoassay (PerkinElmer Life Sciences), using the appropriate PEG hGH to generate standard curve.

TABLE 4

| | | 40 K Br | 40 K Br | 30 K | 20 K | 4-6 × |
|---|---|---|---|---|---|---|
| Species | Parameters | ALD hGH | NHS hGH | ALD hGH | ALD hGH | 5 K SPA |
| mouse | dose | iv 1.0 | iv 1.0 | iv 1.0 | iv 1.0 | iv 1.0 |
| | (mg/kg) | sc 1.8 | sc 1.8 | Sc 1.8 | Sc 1.0 | sc 1.8 |
| | CL | 2.29 | 2.12 | 4.43 | 7.89 | 4.53 |
| | (ml/hr/kg) | | | | | |
| | Vss | 18 | 16 | 24 | 17 | 51 |
| | (ml/kg) | | | | | |
| | T_{½}, iv | 4.3 | 3.8 | 2.8 | 1.8 | 11 |
| | (hr) | | | | | |
| | T_{½}, sc | 4 | 6.2 | 3.7 | 2.5 | 9 |
| | (hr) | | | | | |
| | Tmax, sc | 11 | 9 | 6 | 3 | 12 |
| | (hr) | | | | | |
| | SC AUC | 682 | 577 | 160 | 31 | 668 |
| | (ug/ml * hr) | | | | | |
| | SC Bioavailability | 87 | 67 | 39 | 24 | 167 |
| | (%) | | | | | |
| rat | dose | iv 1.0 | iv 1.0 | iv 1.0 | iv 1.8 | iv 1.0 |
| | (mg/kg) | sc 1.8 | sc 1.8 | sc 1.8 | sc 1.8 | sc 1.8 |
| | CL | 1.36 | 1.75 | 5.75 | 9.9 | 2.9 |
| | (ml/hr/kg) | | | | | |
| | Vss | 19 | 25 | 44 | 33 | 36 |
| | (ml/kg) | | | | | |
| | T_{½}, iv | 5.4 | 5.8 | 3.6 | 2.2 | 24 |
| | (hr) | | | | | |
| | T_{½}, sc | 5.8 | 7.1 | 6.7 | 2.9 | 29 |
| | (hr) | | | | | |
| | Tmax, sc | 24 | 22 | 12 | 9 | 20 |
| | (hr) | | | | | |
| | SC AUC | 398 | 344 | 97 | 70 | 249 |
| | (ug/ml * hr) | | | | | |
| | SC Bioavailability | 30 | 33 | 31 | 39 | 40 |
| | (%) | | | | | |
| cyno | dose | iv 0.18 | iv 0.18 | iv 0.18 | iv 0.18 | iv 0.18 |
| | (mg/kg) | sc 0.18 | sc 0.18 | sc 0.18 | sc 0.18 | sc 0.18 |
| | CL | 1.83 | 0.78 | 1.94 | 2.19 | 0.49 |
| | (ml/hr/kg) | | | | | |
| | Vss | 57 | 20 | 29 | 44 | 25 |
| | (ml/kg) | | | | | |
| | T_{½}, iv | 21 | 13.6 | 14.9 | 7.3 | 38 |
| | (hr) | | | | | |
| | T_{½}, sc | 19 | 21 | 12 | 8.3 | 35 |
| | (hr) | | | | | |
| | Tmax, sc | 22 | 22 | 10 | 8 | 32 |
| | (hr) | | | | | |
| | SC AUC | 100 | 483 | 125 | 38 | 242 |
| | (ug/ml * hr) | | | | | |
| | SC Bioavailability | 64 | 77 | 97 | 44 | 66 |
| | (%) | | | | | |

What is claimed is:

1. A conjugate comprising at least one water-soluble polymer molecule covalently attached to at least one amino acid residue of a biologically active human growth hormone (hGH) polypeptide or agonist variant thereof.

2. The conjugate of claim 1 wherein said hGH polypeptide comprises the amino acid sequence of SEQ ID NO: 1.

3. The conjugate of claim 1 or 2, wherein said polymer is selected from a group consisting of poly(alkylene oxide), poly(oxyethylated polyol), poly(vinylalcohol), poly(olefenic alcohol), poly(acryloyl morpholine), poly(oxazoline), poly(vinyl pyrrolidone), poly(hydroxyethyl methacrylate), dextran, and derivatives thereof.

4. The conjugate of claim 3 wherein said poly(alkylene oxide) molecule is a poly(ethylene glycol) molecule.

5. The conjugate of claim 4 wherein the poly(ethylene glycol) is attached to said polypeptide at an amino acid residue having a free amino group(s), carboxyl group(s), or sulfhydryl group(s).

6. The conjugate of claim 5 formed using an activated poly(ethylene glycol).

7. The conjugate of claim 6 wherein said activated poly(ethylene glycol) comprises a reactive functional group.

8. The conjugate of claim 7 wherein said attachment is at an amino acid of said polypeptide having a free amino group.

9. The conjugate of claim 8 wherein said reactive functional group is selected from the group consisting of: carbonate esters, active esters of carboxylic acids, dichlorotriazine, tresylate, azlactones, cyclic imidethiones, isocyanates or isothiocyanates, imidates, thioimidates, carbonyldiimidazole, aldehydes, aldehyde hydrates, acid chloride, and carboxylic acid wherein said carboxylic acid is in the presence of activating agent is selected from the group consisting of dicyclohexylcarbodiimide, N-(dimethylaminopropyl)-N′-ethylcarbodiimide, diphenylphosphorylazide, isobutylchloroformate, and ethylchloroformate.

10. The conjugate of claim 9 wherein said reactive functional group is a carbonate ester or carbonyldimidazole.

11. The conjugate of claim 10 wherein said activated poly(ethylene glycol) is selected from the group consisting of:

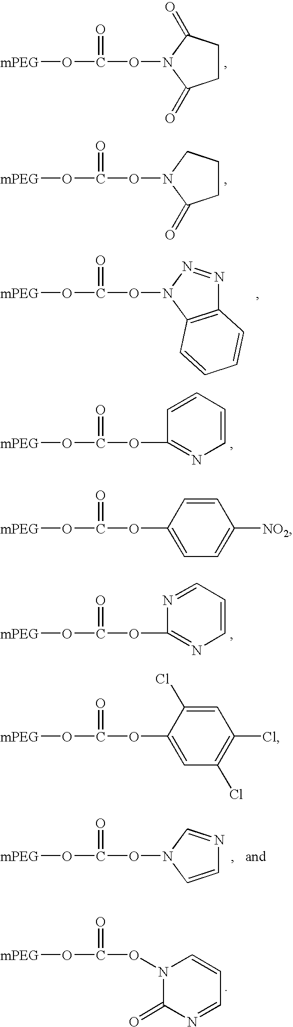

12. The conjugate of claim 11 of the formula 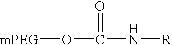
wherein R is a human growth hormone polypeptide.

13. The conjugate of claim 12 wherein said human growth hormone polypeptide comprises the amino acid sequence of SEQ ID NO: 1.

14. The conjugate of claim 9 wherein said reactive functional group is a cyclic imidethione.

15. The conjugate of claim 14 wherein said activated poly(ethylene glycol) is selected from the group consisting of: 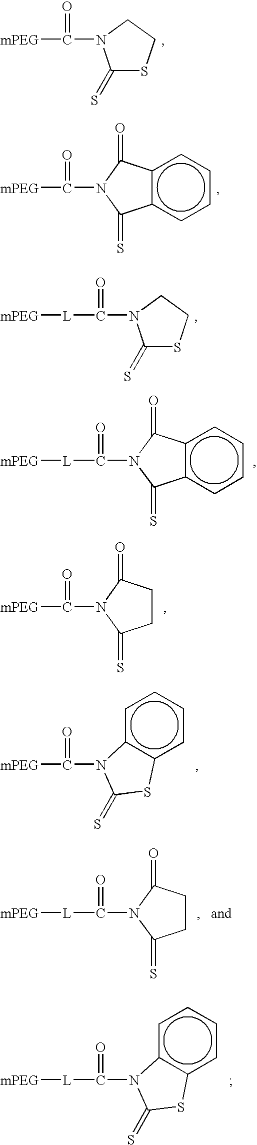
wherein L is selected from the group consisting of: —O—, —NH—, —OCH₂—, —NH—CO(CH₂)ₙ, —NH—CO(CH₂)ₙO—, —CO—NH(CH₂)ₙ—, —S—, —CO—NH(CH₂)ₙO—, —O(CH₂)_{N}O—, —O(CH₂)ₙ—, —SCH₂CH₂—, and —NH(CH₂)ₙ—.

16. The conjugate of claim 9 wherein said reactive functional group is an azlactone.

17. The conjugate of claim 16 wherein said activated poly(ethylene glycol) is selected from the group consisting of: 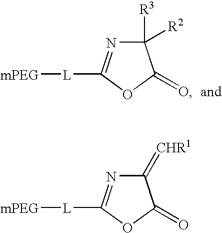
wherein
R¹ is selected from the group consisting of hydrogen, alkyl, cycloalkyl, carbocyclic and heterocyclic aromatic rings, α, β-unsaturated alkyl; and
R² and R³ are independently selected from hydrogen, alkyl, aryl, and alkylaryl.

18. The conjugate of claim 9 wherein said functional group is a isocyanate or isothiocyanate.

19. The conjugate of claim 18 wherein said activated poly(ethylene glycol) is selected from the group consisting of:
mPEG-N═C═O, and mPEG-N═C═S.

20. The conjugate of claim 19 of the formula selected from the group consisting of: 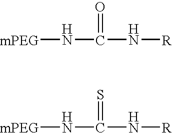
wherein R is a human growth hormone polypeptide.

21. The conjugate of claim 9 wherein said functional group is an aldehyde, acetal aldehyde or aldehyde hydrate.

22. The conjugate of claim 21 wherein said activated poly(ethylene glycol) is selected from the group consisting of:

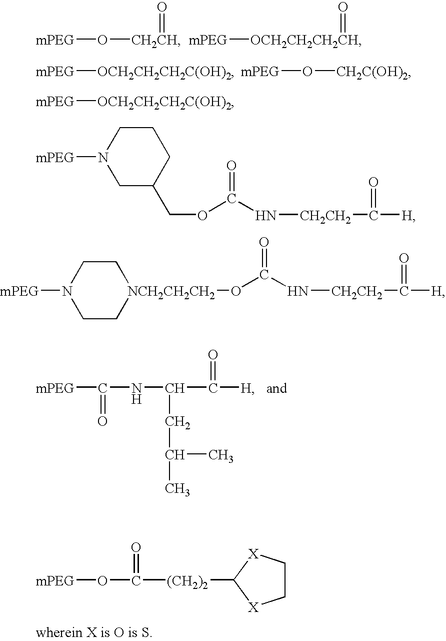

23. The conjugate of claim 22 of the formula selected from the group consisting of: 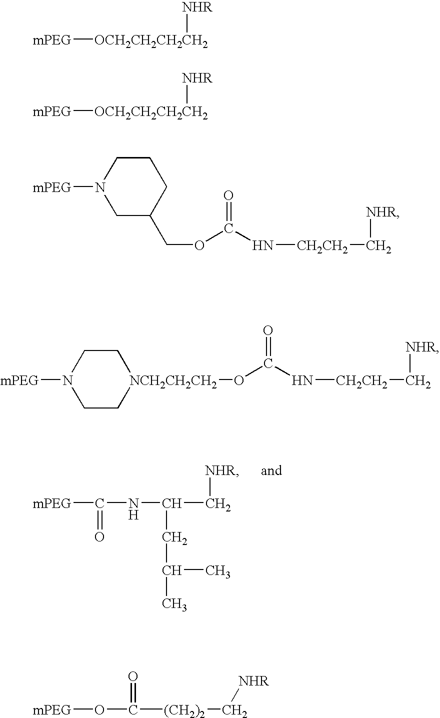
wherein R is a human growth hormone polypeptide.

24. The conjugate of claim 9 wherein said reactive functional group is an active ester of a carboxylic acid.

25. The conjugate of claim 24 wherein said activated poly(ethylene glycol) is selected from the group consisting of:

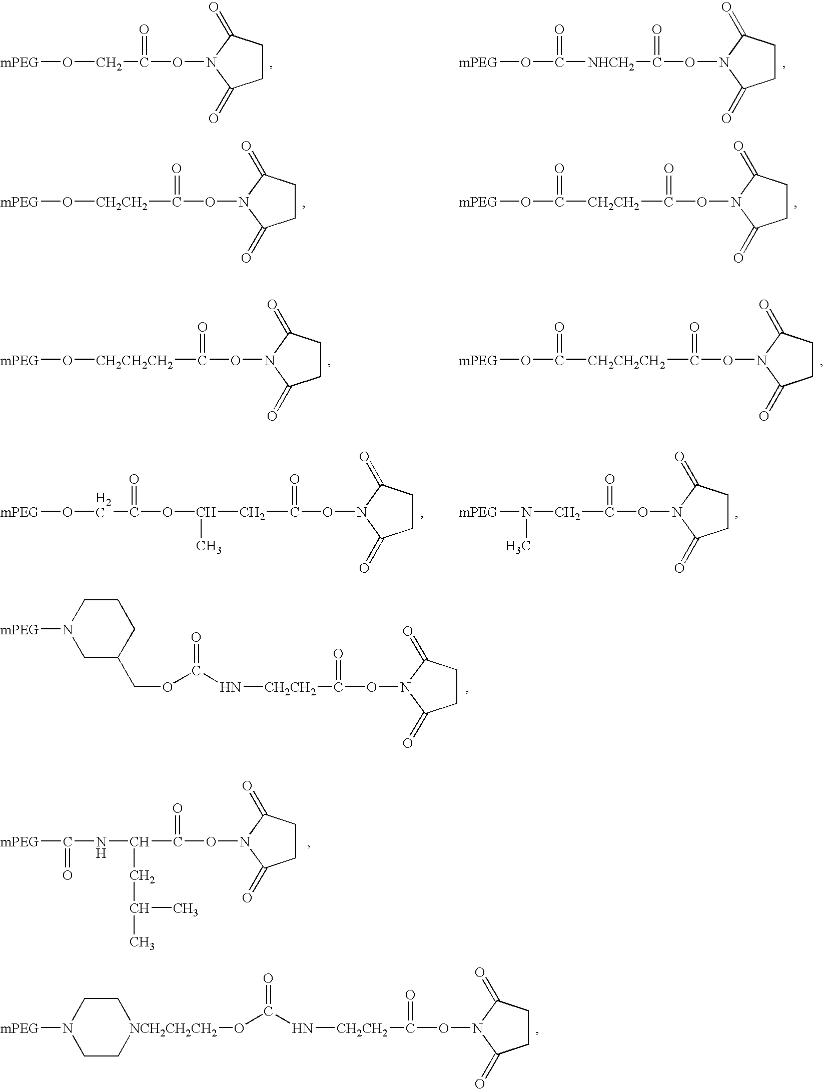

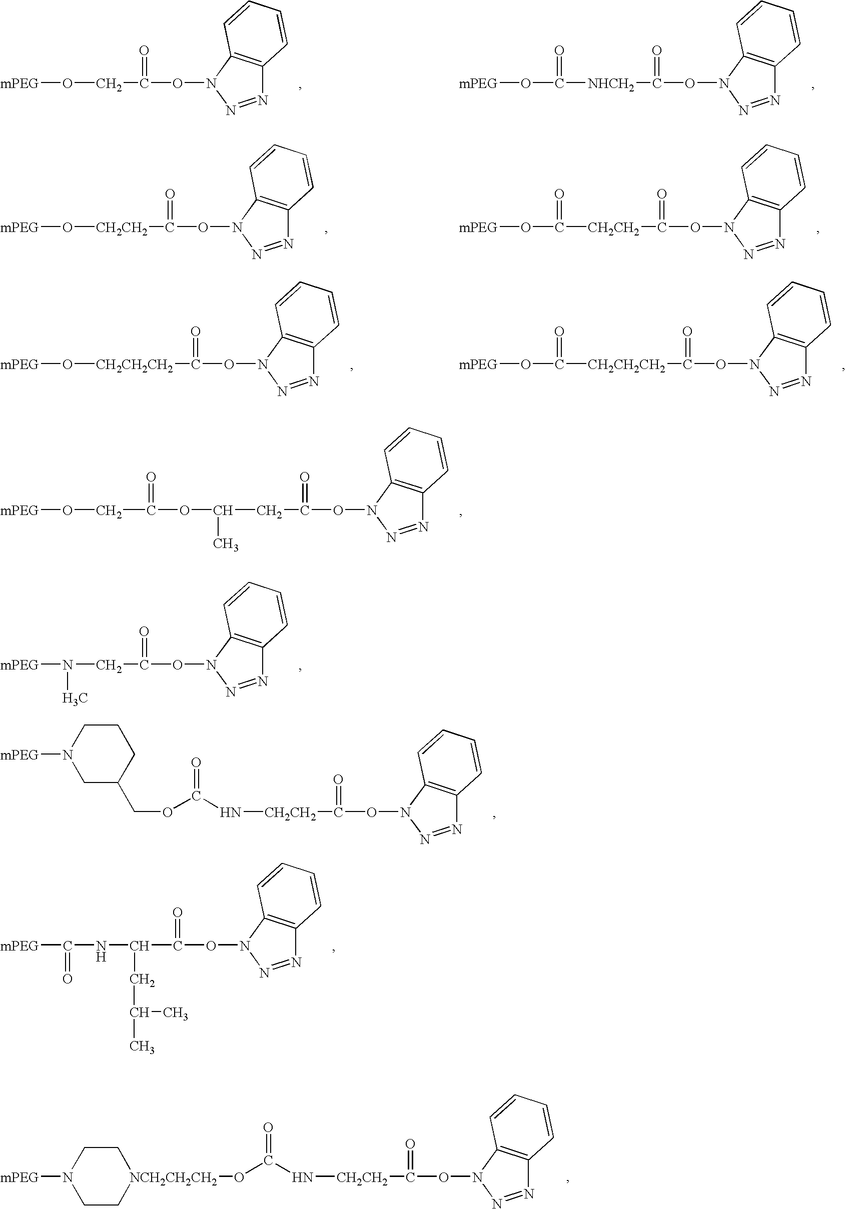

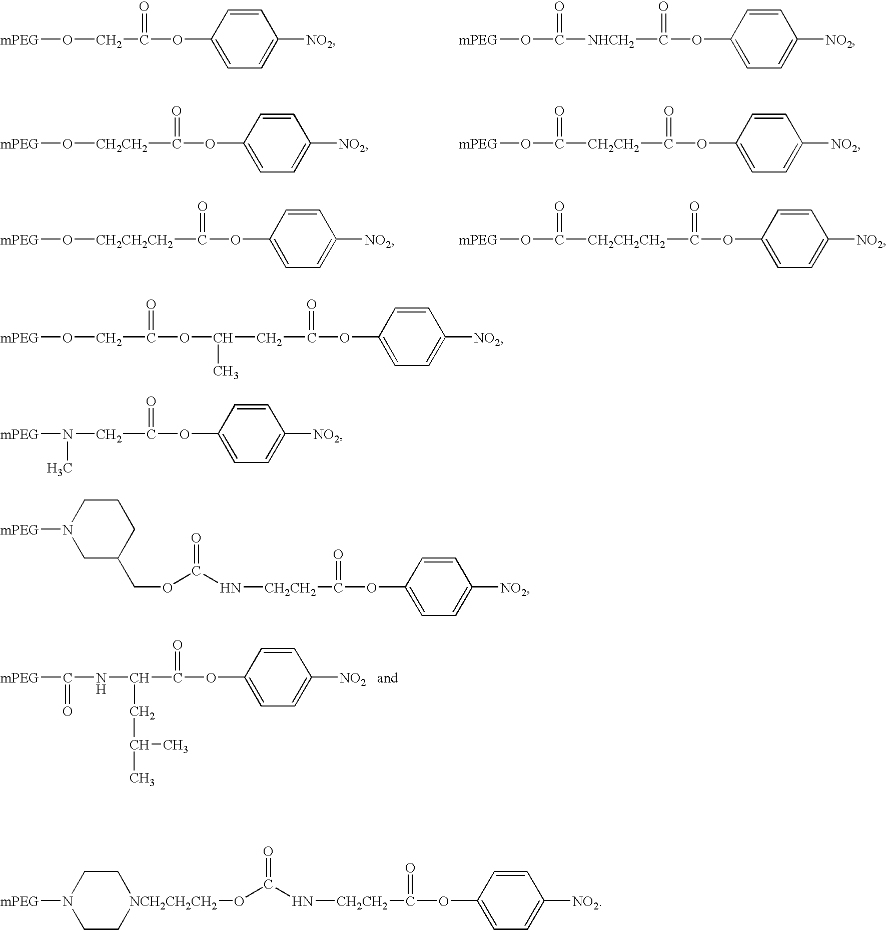

26. The conjugate of claim 25 of the formula selected from the group consisting of: 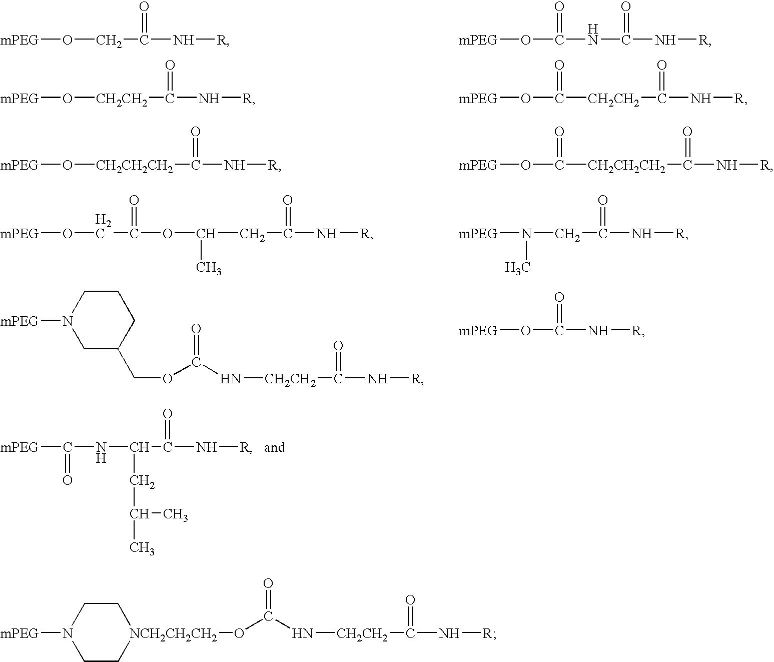
wherein R is a human growth hormone polypeptide.

27. The conjugate of claim 26 wherein said human growth hormone polypeptide comprises the amino acid sequence of SEQ ID NO: 1.

28. The conjugate of claim 9 wherein said activated poly(ethylene glycol) is selected from the group consisting of:
mPEG-O—SO₂—CH₂CF₃, and

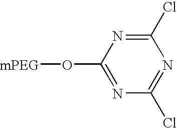

29. The conjugate of claim 28 of the formula selected from the group consisting of:
PEG-NH—R, and 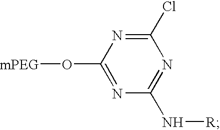
wherein R is a human growth hormone polypeptide.

30. The conjugate of claim 29 wherein said human growth hormone polypeptide comprises the amino acid sequence of SEQ ID NO: 1.

31. The conjugate of claim 9 wherein said reactive functional group is an imidate or thioimidate.

32. The conjugate of claim 31 wherein said activated poly(ethylene glycol) is selected from the group consisting of:

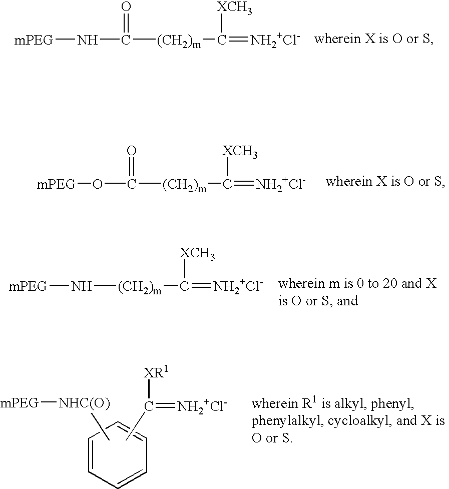

33. The conjugate of claim 32 of the formula selected from the group consisting of:

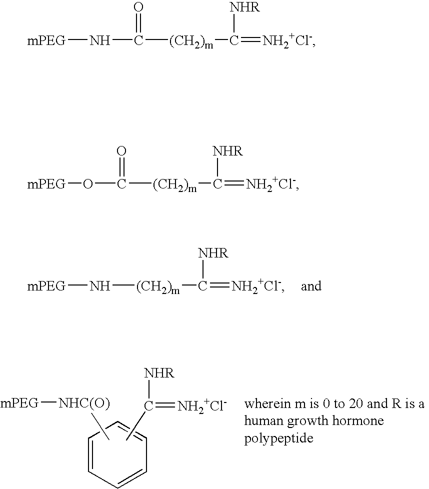

34. The conjugate of claim 8 wherein said free amino group is an amino terminal α-amino group.

35. The conjugate of claim 34 wherein said amino terminal α-amino group is on a phenylalanine.

36. The conjugate of claim 8 wherein said attachment is at an amino acid of said polypeptide having a free carboxyl group.

37. The conjugate of claim 36 wherein said reactive functional group is selected from the group consisting of: primary amines, hydrazine, and hydrazide groups.

38. The conjugate of claim 36 wherein said reactive functional group is selected from the group consisting of:

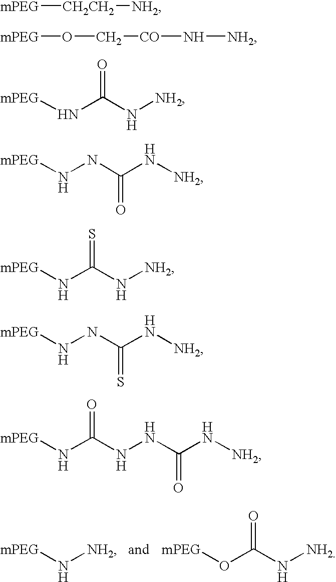

39. The conjugate of claim 8 wherein said attachment is at an amino acid of said polypeptide having a free sulfhydryl group.

40. The conjugate of claim 39 wherein said reactive functional group is selected from the group consisting of: thiols, maleimides, vinyl sulfones, iodoacetamide and phenyl glyoxals.

41. The conjugate of claim 40 wherein said reactive functional group is selected from the group consisting of:

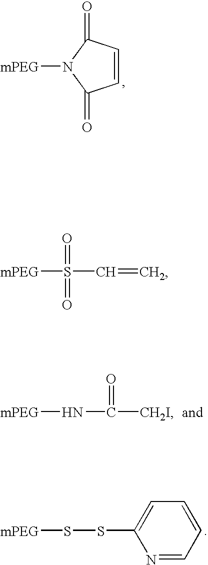

42. The conjugate of claim 41 of the formula selected from the group consisting of: 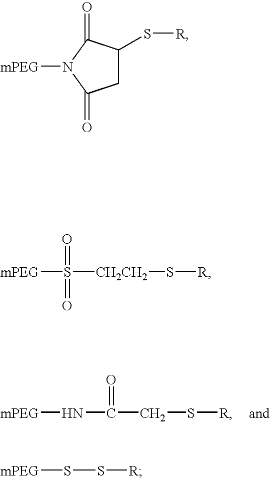
wherein R is a human growth hormone polypeptide.

43. The conjugate of claim 42 wherein said human growth hormone polypeptide comprises the amino acid sequence of SEQ ID NO: 1.

44. The conjugate of claim 8 wherein said poly(ethylene glycol) has a molecular weight of between about 0.5 kDa and about 100 kDa.

45. The conjugate of claim 44 wherein said poly(ethylene glycol) has a molecular weight of between about 5 kDa and about 40 kDa.

46. The conjugate of claim 8 wherein said poly(ethylene glycol) is a branched polymer.

47. The conjugate of claim 46 wherein said branched polymer is selected from the group consisting of:

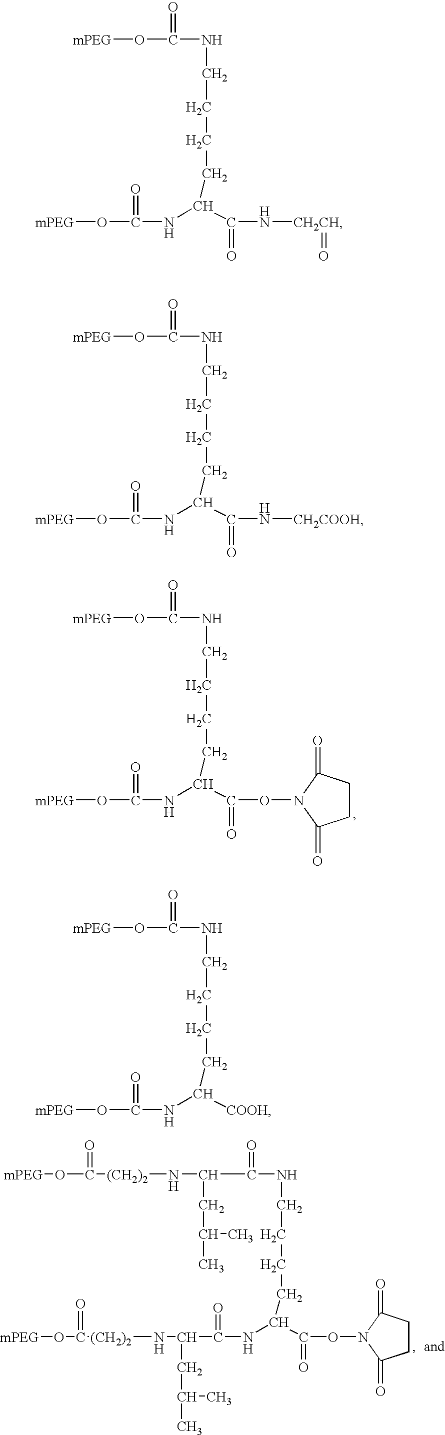

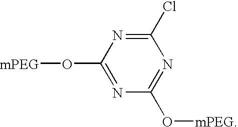

48. A human growth hormone-PEG conjugate of the formula 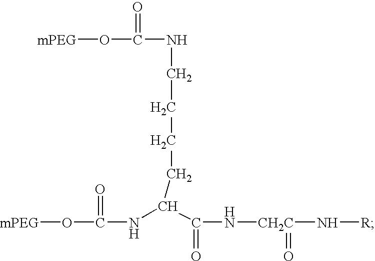
wherein R is a human growth hormone polypeptide.

49. The conjugate of claim 48 wherein said human growth hormone polypeptide comprises the amino acid sequence of SEQ ID NO: 1.

50. The conjugate of claim 49 wherein at least 80% of said poly(ethylene glycol) is conjugated to the amino-terminal phenylalanine.

51. The conjugate of claim 50 wherein at least 90% of said poly(ethylene glycol) is conjugated to the amino-terminal phenylalanine.

52. The conjugate of claim 51 or 52 wherein each mPEG has an average molecular weight of about 20 kDa.

53. A human growth hormone-PEG conjugate of the formula 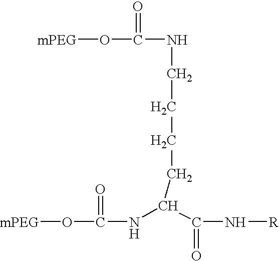
wherein R is a human growth hormone polypeptide.

54. The conjugate of claim 53 wherein said human growth hormone polypeptide comprises the amino acid sequence of SEQ ID NO: 1.

55. The conjugate of claim 53 wherein at least 80% of said poly(ethylene glycol) is conjugated to the amino-terminal phenylalanine.

56. The conjugate of claim 53 wherein at least 90% of said poly(ethylene glycol) is conjugated to the amino-terminal phenylalanine.

57. The conjugate of claim 54 or 55 wherein each mPEG has a molecular weight of about 20 kDa.

58. A human growth hormone-PEG conjugate of the formula
mPEG-OCH₂CH₂CH₂NH—R
wherein R is a human growth hormone polypeptide.

59. The conjugate of claim 58 wherein said human growth hormone polypeptide comprises the amino acid sequence of SEQ ID NO: 1.

60. The conjugate of claim 58 wherein at least 80% of said poly(ethylene glycol) is conjugated to the amino-terminal phenylalanine.

61. The conjugate of claim 58 wherein at least 90% of said poly(ethylene glycol) is conjugated to the amino-terminal phenylalanine.

62. The conjugate of claim 59 or 60 wherein each mPEG has an average molecular weight of about 20 kDa.

63. The conjugate of claim 7 wherein said poly(ethylene glycol) is a bifunctional polymer.

64. The conjugate of claim 7 wherein said poly(ethylene glycol) is a prodrug.

65. A composition comprising the hGH conjugate of claim 1 and at least one pharmaceutically acceptable carrier.

66. A method of treating a patient having a growth or development disorder or comprising administering to said patient a therapeutically effective amount of the hGH conjugate of claim 1.

67. The method of claim 66 wherein said growth or development disorder is Growth Hormone Deficiency (GHD).

68. The method of claim 66 wherein said growth or development disorder is Turner's syndrome.

69. The method of claim 66 wherein said growth or development disorder is Chronic Renal Insufficiency.

70. The method of claim 66 wherein said growth or development disorder is small for gestational age (SGA).

71. A composition comprising the hGH conjugate of claim 51 and at least one pharmaceutically acceptable carrier.

72. A method of treating a patient having a growth or development disorder or comprising administering to said patient a therapeutically effective amount of the hGH conjugate of claim 51.

73. The method of claim 72 wherein said growth or development disorder is Growth Hormone Deficiency (GHD).

74. The method of claim 72 wherein said growth or development disorder is Turner's syndrome.

75. The method of claim 72 wherein said growth or development disorder is Chronic Renal Insufficiency.

76. The method of claim 72 wherein said growth or development disorder is small for gestational age (SGA).

77. A composition comprising the hGH conjgate of claim 53 or 58 and at least one pharmaceutically acceptable carrier.

78. A method of treating a patient having a growth or development disorder or comprising administering to said patient a therapeutically effective amount of the hGH conjugate of claim 53 or 58.

79. The method of claim 78 wherein said growth or development disorder is Growth Hormone Deficiency (GHD).

80. The method of claim 78 wherein said growth or development disorder is Turner's syndrome.

81. The method of claim 78 wherein said growth or development disorder is Chronic Renal Insufficiency.

82. The method of claim 78 wherein said growth or development disorder is small for gestational age (SGA).
